SEQUENCE LISTING (4 sequences)

SEQ ID NO: 1 - protein, 40 aa, Mus musculus
YGTRKYKKKEYQEIEEQESLVGSLATDATVLNGEEDPSPA

SEQ ID NO: 2 - protein, 41 aa, Canis familiaris
YGTRKYKKRGYQALEDERQSLAAGTAESGDAPGQEQEQSPA

SEQ ID NO: 3 - protein, 41 aa, Equus caballus
YGTRKYKKREYQALEEERQSLAPGTAETRDMPDQEQEQSPA

SEQ ID NO: 4 - protein, 40 aa, Homo sapiens
YGTRKFKKKAYQAIEERQSLVPGTAATGDTTYQEQGQSPA

(19) United States
(12) Patent Application Publication
(10) Pub. No.: US 2016/0250303 A1
(43) Pub. Date: Sep. 1, 2016

(54) PERFORIN-2 ACTIVATORS AND INHIBITORS AS DRUG TARGETS FOR INFECTIOUS DISEASE AND GUT INFLAMMATION

(71) Applicant: UNIVERSITY OF MIAMI, Miami, FL (US)

(72) Inventors: Eckhard R. Podack, Coconut Grove, FL (US); Ryan McCormack, Charlotte, NC (US)

(21) Appl. No.: 15/028,217

(22) PCT Filed: Oct. 8, 2014
(86) PCT No.: PCT/US2014/059675

§ 371 Date: Apr. 8, 2016

Related U.S. Application Data
(60) Provisional application No. 61/888,919, filed on Oct. 9, 2013.
(60) Provisional application No. 61/927,591, filed on Jan. 15, 2014.

(51) Int. Cl. A61K 38/50; A61K 31/519

(52) U.S. Cl. CPC A61K 38/50; A61K 31/519; C12Y 305/00

(57) ABSTRACT

Methods and compositions are provided to modulate the activity of Perforin-2. Provided herein are various components of the Perforin-2 activation pathway. In specific embodiments, inhibitors of the various components of the Perforin-2 activation pathway are provided which may be employed in various methods, including, but not limited to, the diagnosis and treatment of diseases associated with gut inflammation. Methods of screening for Perforin-2 inhibitors are also provided. Further provided are compounds that increase the ubiquitination of Perforin-2 and thereby increase Perforin-2 activity. Various methods for increasing Perforin-2 activity and for the treatment of infectious disease, in particular bacteria and antibiotic-resistant bacteria, are also provided.

### REFERENCE TO A SEQUENCE LISTING SUBMITTED AS A TEXT FILE VIA EFS-WEB

The official copy of the sequence listing is submitted concurrently with the specification as a text file via EFS-Web, in compliance with the American Standard Code for Information Interchange (ASCII), with a file name of 452788seq1ist.txt, a creation date of Oct. 7, 2014 and a size of 2 KB. The sequence listing filed via EFS-Web is part of the specification and is hereby incorporated in its entirety by reference herein.

### FIELD OF THE INVENTION

This invention relates to the fields of infectious disease and gut inflammation.

### BACKGROUND OF THE INVENTION

Perforin is a cytolytic protein found in the granules of CD8 T-cells and NK cells. Upon degranulation, perforin inserts itself into the target cell's plasma membrane, forming a pore. The cloning of Perforin by the inventors' laboratory (Lichtenheld, M. G., et al., 1988. Nature 335:448-451; Lowrey, D. M., et al., 1989. Proc Natl Acad Sci USA 86:247-251) and by Shinkai et at (Nature (1988) 334:525-527) established the postulated homology of complement component C9 and of perforin (DiScipio, R. G., et al., 1984. Proc Natl Acad Sci USA 81:7298-7302).

Both Perforin-1 and Perforin-2 (P2) are pore formers that are synthesized as hydrophilic, water soluble precursors. Both can insert into and polymerize within the lipid bilayer to form large water filled pores spanning the membrane. The water filled pore is made by a cylindrical protein-polymer.

The inside of the cylinder must have a hydrophilic surface because it forms the water filled pore while the outside of the cylinder needs to be hydrophobic because it is anchored within the lipid core. This pore structure is thought to be formed by an amphipathic helix (helix turn helix). It is this part of the protein domain, the so called MAC-Pf (membrane attack complex/Perforin) domain, that is most conserved between Perforin and C9 and the other complement proteins forming the membrane attack complex (MAC) of complement.

An mRNA expressed in human and murine macrophages (termed Mpg 1 or Mpeg 1-macrophage expressed gene) predicting a protein with a MAC/Pf domain was first described by Spilsbury (Blood (1995) 85:1620-1629). Subsequently, the same mRNA (named MPS-1) was found to be upregulated in experimental prion disease. The group of Desjardin analyzed the protein composition of phagosome membranes isolated from macrophages fed with latex beads by 2D-gel electrophoresis and mass spectrometry (J Cell Biol 152:165-180, 2001). The authors found protein spots corresponding to the MPS-1 protein. Mah et al analyzed abalone mollusks and found an mRNA in the blood homologous to the Mpeg1 gene family (Biochem Biophys Res Commun 316:468-475, 2004) and suggested that predicted protein has similar functions as CTL perforin but that it is part of the innate immune system of mollusks.

Multidrug resistance is the ability of pathologic cells to withstand chemicals that are designed to aid in the eradication of such cells. Pathologic cells include but are not limited to fungal, bacterial, virally infected and neoplastic (tumor) cells. Many different bacteria now exhibit multidrug resistance, including staphylococci, enterococci, gonococci, streptococci, salmonella and others. Additionally, some resistant bacteria are able to transfer copies of DNA that codes for a mechanism of resistance to other bacteria, thereby conferring resistance to their neighbors, who then are also able to pass on the resistant gene.

Bacteria have been able to adapt to antibiotics by e.g., no longer relying on glycoprotein cell wall; enzymatic deactivation of antibiotics; decreased cell wall permeability to antibiotics; or altered target sites of antibiotic efflux mechanisms to remove antibiotics. As such, there is a growing need for overcoming multi-drug resistance by way of new drugs that attack pathological cells in new ways.

### SUMMARY OF THE INVENTION

Methods and compositions are provided to modulate the activity of Perforin-2. Provided herein are various components of the Perforin-2 activation pathway. In specific embodiments, inhibitors of the various components of the Perforin-2 activation pathway are provided which may be employed in various methods, including, but not limited to, the diagnosis and treatment of diseases associated with gut inflammation. Methods of screening for Perforin-2 inhibitors are also provided. Further provided are compounds that increase the ubiquitination of Perforin-2 and thereby increase Perforin-2 activity. Various methods for increasing Perforin-2 activity and for the treatment of infectious disease, in particular bacteria and antibiotic-resistant bacteria, are also provided.

### BRIEF DESCRIPTION OF THE DRAWINGS

FIG. 1 shows clustered poly-Perforin-2 pores/holes (100 Å) seen by electron microscopy on membrane fragments of (a) eukaryotic cells, (b) M. smegmatis, (c) S. aureus (MRSA). White arrows point to single Perforin-2 polymers, black arrows point to clusters of Perforin-2 polymers.

FIG. 2 depicts the structure and orientation of Perforin-2 (P-2) in cytosolic vesicles. Also depicted is the Perforin-2 domain structure and conservation of the cytoplasmic domain.

FIG. 3 shows that P-2-GFP translocates to the SCV. Microglia BV2 were transfected with P-2-GFP, infected with Salmonella typhimurium and fixed 5 min after infection and imaged. Please note the translocation of P-2-GFP from the cytosol in uninfected cells to the SCV and release of DNA from the rod like Salmonella (arrow, Salmonella outside the cell), suggesting killing by P-2.

FIG. 4 depicts Perforin-2 interacting proteins for translocation and polymerization. For clarity, only one Perforin-2 molecule is shown—many polymerize and refold inserting the β-hairpins.

FIG. 5 depicts pathways of neddylation and deneddylation that control Perforin-2 ubiquitination, ploymerization and bacterial killing. NAE=NEDD8 activating enzyme.

FIG. 6 shows genetically P-2 deficient or P-2 siRNA depleted peritoneal macrophages are unable to prevent intracellular Salmonella replication.

FIG. 7 shows that P-2 knock-down enables intracellular bacterial replication in PMN (upper panels) and rectal epithelial cells. P-2-GFP overexpression increases bactericidal activity (lower panels).

FIG. 8 demonstrates that ROS and NO contribute to bactericidal activity only in the presence of P-2, but not in P-2 knock-down as shown by NAC and NAME inhibition. Filled symbols: P-2 siRNA knock down. Open symbols: scramble siRNA controls (P-2 present).

FIG. 9 shows that P-2 deficient mice succumb to epicutaneous MRSA challenge. P-2−/−, P-2+/− and P-2+/+ litter mates (7 per group) were shaved (2×2 cm) tape stripped 7 times, infected with 1 cm² filter disk soaked with 10⁷ MRSA, clinical isolate. Weight (left panel) and cfu in various organs and blood on day 6.

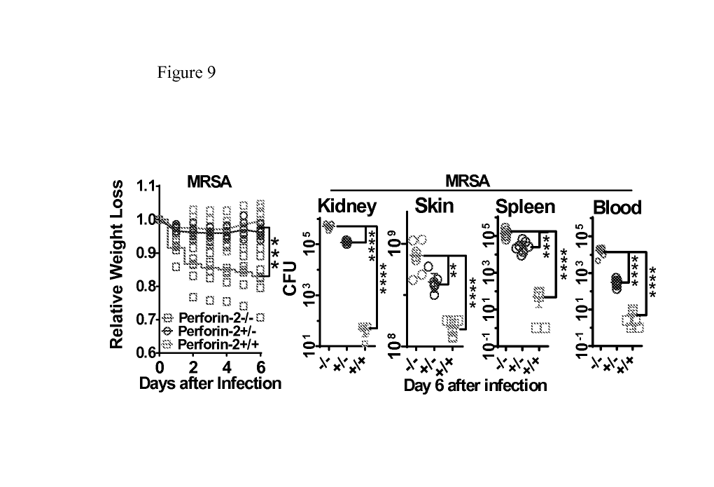

FIG. 10 demonstrates that P-2−/− mice die from orogastric infection with 10⁵ or 10² S. typhimurium that are cleared in P-2+/+ and +/− littermates. n=8 or 15 per group.

FIG. 11 depicts P-2−/− mice have high level cfu in blood and other organs after orogastric S. typhimurium infection.

FIG. 12 shows minimal inflammation in P-2−/− mice challenged with S. Typhimurium despite high cfu.

FIG. 13 shows that P-2−/− mice are resistant to DSS colitis. 3% DSS in water was given for 5 days and then replaced by normal water.

FIGS. 14 A and B shows, in a larger group of mice, resistance to DSS colitis if they are Perforin-2 deficient. (C) Perforin-2 mediated killing of MRSA by the phagocytic cell BV2 is blocked by the chemical drug MLN4294 indicating involvement of NEDD8 in Perforin-2 activation.

FIG. 15 shows (a) Induction of Perforin-2 mRNA in murine embryonic fibroblasts by IFN-α,β,γ; (b) Constitutive Perforin-2 protein expression in peritoneal macrophages.

FIG. 16 shows Perforin-2 mRNA induction in MEF by IFN-γ, non-pathogenic E. coli K12 and heat killed Salmonella. Suppression of induction of Perforin-2 by live Salmonella and other pathogens listed.

FIG. 17 shows Perforin-2 expression and killing. Top: Kinetics of Perforin-2 mRNA induction in MEF after intracellular infection with non-pathogenic E. coli K12 and M. smegmatis. 1 h infection at MoI 50:1 and then washing and plating in membrane impermeant gentamicin. Bottom: Kinetics of intracellular killing of M. smegmatis in uninduced MEF (open squares) or induced with IFN-γ for 14 h (filled circles). Note correlation of killing by 12 h with Perforin-2 mRNA expression in uninduced cells.

FIG. 18 shows Perforin-2 knock-down enables M. smegmatis to replicate intracellularly and kill the host cell (columnar epithelium). Control scramble siRNA does not affect Perforin-2 levels and the cells reject M. smegmatis.

FIG. 19 shows Perforin-2 deficient macrophages and PMN are unable to kill intracellular Mtb (a) Mtb (mCherry-Mtb, CDC1551, reporter bacteria) replicate significantly faster in IFN-γ and LPS activated, Perforin−/− than +/+ or +/− bone marrow derived macrophages; (b) M. avium replicates significantly faster in Perforin-2−/− than +/+ or +/−PMN. (c) Perforin-2 is required by PMN to kill M. smegmatis, MRSA and Salmonella. (d) M. tuberculosis CDC1551 was engineered to express mCherry constitutively as a correlate of bacterial survival/growth.

FIG. 20 depicts a model of P-2 vesicle translocation, membrane fusion and pore formation in the bacterial envelop. BCV/SCV=bacterium/salmonella containing vacuole. Red circle with black center is polymerized Perforin-2.

FIG. 21 depicts the crystal structure of Perforin-1 and models of Perforin-1 and -2. (a) Monomeric Perforin-1. The domains are labeled in the cartoon below. Note the CH1 and CH2 parts of the MACPF-domain refolding to β-hairpins in polymerized Perforin-1 and inserting into the membrane. (b) A monomer within polymerized Perforin-1 with β-hairpins inserted into a lipid bilayer. (c) Model of Perforin-2 tethered to the phagosome membrane with the MACPF domain attacking a bacterium inside the phagosome.

FIG. 22 demonstrates that Perforin-2-GFP and RASA2/GAP1M colocalizes with the Salmonella Containing Vacuole (Left panel). Right panel: Perforin-2-RFP colocalizes with the GFP-E. coli containing vacuole.

FIG. 23 shows Perforin-2 interacting proteins by coimmunoprecipitation. RAW cells were transfected with GFP or Perforin-2-GFP and immunoprecipitated with anti-GFP (antibodies to detect and precipitate native Perforin-2 are not available), and the immunoprecipitates blotted with the indicated antibodies.

FIG. 24 shows that Cif deficient Yersinia pseudotuberculosis are sensitive to Perforin-2 killing by endogenous Perforin-2 or by complemented Perforin-2-GFP. (a) Yersinia pseudotuberculosis (Y.pt) is protected from Perforin-2 by chromosomal Cif; (b) Deletion of Cif makes Y.pt sensitive to Perforin-2. Knock-down of Perforin-2 is complemented with Perforin-2-GFP; (c) Cif plasmid protects Y.pt against endogenous Perforin-2 and complemented Perforin-2-GFP.

FIG. 25 demonstrates lysates of killed Yersinia blotted with anti-Perforin-2 show a new Perforin-2 fragment band not detected when Cif is present and the bacteria survive. Perforin-2-GFP immunoprecipitates (with anti GFP) are ubiquitin-negative when killing is blocked by Cif and ubiquitin positive when Cif is absent and the bacteria are killed. Yersinia pseudotuberculosis contained endogenous chromosomal Cif or were Cif deleted and reconstituted and incubated with Perfroin-2-GFP transfected CMT93 cells. 4 h time points were analyzed by western blotting of lysates with anti-Perforin-peptide antiserum (Abcam); anti-GFP immunoprecipitation were immunoblotted with anti-ubiquitin.

FIG. 26 shows orogastric challenge of Perforin-2+/+(green), +/−(blue) and −/−(red) mice with 10⁵ and 10² S. typhimurium RL144; weight loss—upper; survival—lower panels.

FIG. 27 shows (A) the chemical structures of the various inhibitors of E1 ubiquitin-activating enzyme, E2 ubiquitin-conjugating enzyme and E3 ubiquitin ligase provided herein; (B) the chemical structure of a NEDD8 activating enzyme (NAE) inhibitor.

FIG. 28 depicts the chemical structures of the various isopeptidase inhibitors provided herein.

FIG. 29 shows the chemical structures of the various deubiquitinase inhibitors provided herein.

FIG. 30 depicts the chemical structures of the various proteasome inhibitors provided herein.

### DETAILED DESCRIPTION OF THE INVENTION

The present inventions now will be described more fully hereinafter with reference to the accompanying drawings, in which some, but not all embodiments of the invention are shown. Indeed, these inventions may be embodied in many different forms and should not be construed as limited to the embodiments set forth herein; rather, these embodiments are provided so that this disclosure will satisfy applicable legal requirements. Like numbers refer to like elements throughout.

Many modifications and other embodiments of the inventions set forth herein will come to mind to one skilled in the art to which these inventions pertain having the benefit of the teachings presented in the foregoing descriptions and the associated drawings. Therefore, it is to be understood that the inventions are not to be limited to the specific embodiments disclosed and that modifications and other embodiments are intended to be included within the scope of the appended claims. Although specific terms are employed herein, they are used in a generic and descriptive sense only and not for purposes of limitation.

### I. Overview

Methods and compositions are herein provided to modulate the activity of Perforin-2. Modulators of any of the various components of the Perforin-2 activation pathway can be used in the methods and compositions provided herein. In specific embodiments, compounds that inhibit Perforin-2 activity are provided which can be employed in various methods including, but not limited to, the treatment of diseases associated with inflammation of the gut. Compounds that activate Perforin-2 activity are also provided herein and find use in various methods, including, but not limited to, treating diseases caused by an infectious disease organism.

Perforin-2 is expressed constitutively in all phagocytic cells and is inducible in all non-phagocytic cells tested in both mice and humans and plays a role in the killing of pathogenic, intracellular bacteria. Perforin-2 knockdown or deficiency renders cells defenseless and unable to kill intracellular bacteria resulting in intracellular bacterial replication that kills the cells.

Upon polymerization, Perforin-2 forms clusters of large holes and pores in the cell wall/envelop of bacteria that impair the barrier function and permit entry of reactive oxygen and nitrogen species and hydrolases to complete bacterial destruction. Therefore, Perforin-2 is a significant innate effector molecule of unique importance to destroy invading bacteria, particularly antibiotic-resistant bacteria.

As used herein, “Perforin-2 activation pathway” is meant any one or more molecules involved in the modulation of Perforin-2 activity. While not wishing to be limited to a particular mechanism, activation of Perforin-2 comprises at least three steps: (1) Phosphorylation/kinase activation; (2) Translocation of Perforin-2 to bacterium containing membrane; and (3) Polymerization of Perforin-2 resulting in formation of pores in the bacterium surface. Provided herein is the discovery that ubiquitination is a key step for the polymerization and activation of Perforin-2.

Non-limiting examples of the various components of the Perforin-2 activation pathway include, for example: any component of the ubiquitination pathway, ubiquitin, E1 ubiquitin-activating enzyme, E2 ubiquitin-conjugating enzyme, E3 ubiquitin ligase, Cullin ring ubiquitin ligase (CRL), any component of the neddylation pathway, NEDD8, NEDD8 activating enzyme (NAE), deneddylase, deamidase, Ubc12, βTrcP1/2, Skp1, Cullin1, Vps34, RASA2, Ubc4, Rbx1, proteasome, isopeptidases, deubiquitinases, TEC, NEK9, Mapk12, or Perforin-2.

### II. Modulators of Perforin-2 Activity

A series of compounds are provided herein that modulate the activity and/or expression of the various components of the molecular pathway responsible for modulating the activity of Perforin-2. As used herein, the term “modulating” includes “inducing”, “inhibiting”, “potentiating”, “elevating”, “increasing”, “decreasing”, downregulating“, upregulating” or the like. Each of these terms denote a quantitative difference between two states and in particular, refer to at least a statistically significant difference between the two states.

A. Compounds that Inhibit Perforin-2 Activity

Methods and compositions are provided that employ inhibitors of Perforin-2 activity to treat gut inflammation and to treat diseases associated with gut inflammation.

As used herein, “inflammation of the gut” or “gut inflammation” refers to inflammation of the gastrointestinal tract. In some cases, the gut inflammation can be associated with a condition or disease. Non-limiting examples of diseases associated with gut inflammation include, for example, colitis, ulcerative colitis, Crohn's disease or inflammatory bowel disease. In such cases, inhibiting Perforin-2 activity would be beneficial for treating or preventing inflammation of the gut.

Various compounds which inhibit the activity of Perforin-2 are provided herein (i.e. compounds that result in the modulation of any one or more of the various components of the Perforin-2 activation pathway) and thereby act to decrease Perforin-2 activity.

The term “inhibitor” refers to an agent which “reduces”, “inhibits”, “decreases” or otherwise “diminishes” one or more of the biological activities and/or expression of a target (i.e., a target polypeptide or a target signaling pathway) Inhibition using an inhibitor does not necessarily indicate a total elimination of the targeted activity. Instead, the activity could decrease by a statistically significant amount including, for example, a decrease of at least about 5%, 10%, 15%, 20%, 25%, 30%, 35%, 40%, 45%, 50%, 55%, 60%, 65%, 70%, 75%, 80%, 85%, 95% or 100% of the activity of the target compared to an appropriate control.

A decrease in Perforin-2 activity can be assayed in a variety of ways, including, but not limited to, a decrease in the level of Perforin-2 protein by protein expression analysis such as Western blot, immunoprecipitation, immunohistochemistry, immunofluorescence, or a decrease in Perforin-2 mRNA expression by analysis such as Northern blot or RT-PCR. In addition, a decrease in the activity of Perforin-2 can be measured by assaying for a decrease in the bactericidal activity of a cell infected with bacteria. Methods for assaying include, but are not limited to, an increase in bacterial replication, or an increase in cell death of the infected cells. A decrease in Perforin-2 activity can also be measured in vivo by measuring for an increase in bacterial colony forming units in various organs and blood after infection with a bacterium as compared to an appropriate control or through a reduction in inflammation of gut tissue. Various assays to measure Perforin-2 activity are described elsewhere herein.

As used herein, an “inhibitor of Perforin-2 activity” or a “compound that inhibits Perforin-2 activity” refers to a compound that modulates the activity and/or expression of at least one component of the Perforin-2 activation pathway thereby inhibiting Perforin-2, or directly inhibits the activity and/or expression of Perforin-2. In some embodiments, the inhibitor of Perforin-2 activity inhibits the activity of at least one target molecule, thereby inhibiting Perforin-2 activity. In other embodiments, the inhibitor of Perforin-2 activity increases the activity of at least one target molecule, thereby inhibiting Perforin-2 activity.

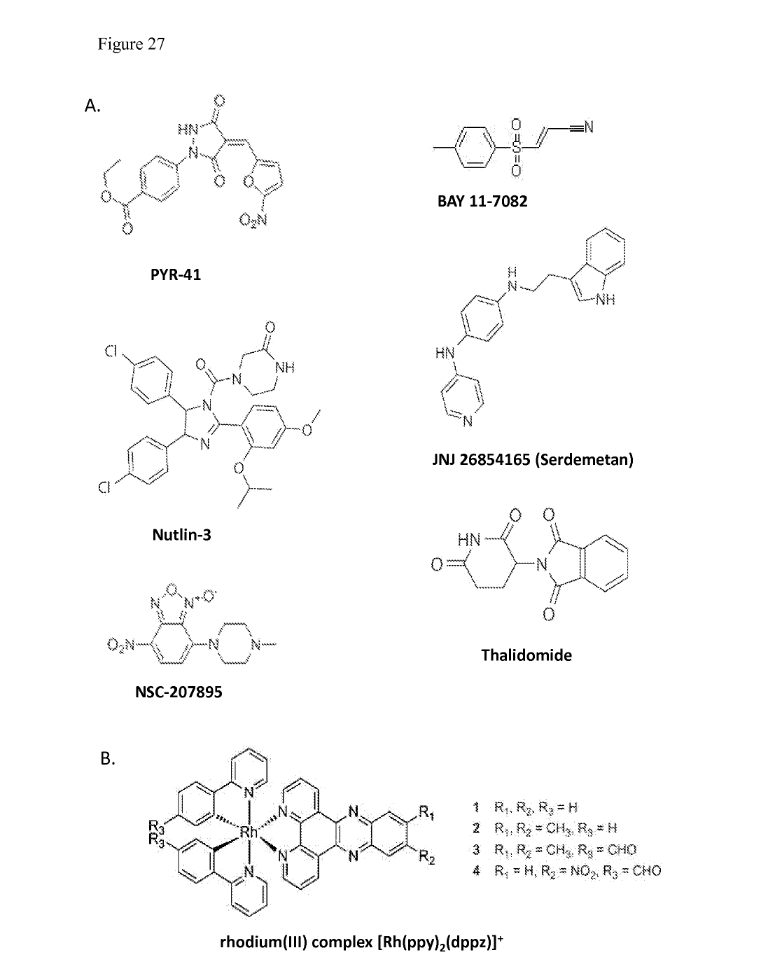

As described in detail elsewhere herein, ubiquitination of Perforin-2 is an important step in Perforin-2 activation. In one embodiment, the compound that inhibits Perforin-2 activity inhibits the ubiquitination of Perforin-2. In certain embodiments, the compound is an inhibitor of at least one component of the ubiquitination pathway. In specific embodiments, the compound that inhibits Perforin-2 activity is an E1 ubiquitin-activating enzyme inhibitor, an E2 ubiquitin-conjugating enzyme inhibitor or an E3 ubiquitin ligase inhibitor. Non-limiting examples of inhibitors of E1 ubiquitin-activating enzyme, E2 ubiquitin-conjugating enzyme or E3 ubiquitin ligase include, for example, PYR-41, BAY 11-7082, Nutlin-3, JNJ 26854165 (Serdemetan), Thalidomide, TAME, NSC-207895, or active derivatives thereof. The chemical structures of the various inhibitors of E1 ubiquitin-activating enzyme, E2 ubiquitin-conjugating enzyme or E3 ubiquitin ligase are shown in FIG. 27A.

As described elsewhere herein, neddylation is a key step in the pathway leading to Perforin-2 activation. In some embodiments, the compound that inhibits Perforin-2 activity is an inhibitor of the neddylation pathway. In some cases, activating a component of the neddylation pathway will result in inhibition of neddylation. In other cases, inhibiting a component of the neddylation pathway will result in inhibition of neddylation. In certain embodiments, the compound is a NEDD8-activating enzyme (NAE) inhibitor.

In some embodiments, the compound that inhibits Perforin-2 activity comprises an NAE inhibitor compound referred to herein as MLN-4924 and comprises the formula:

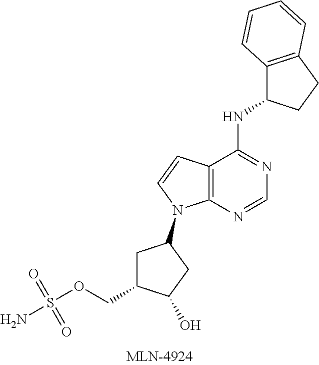

Further provided are active derivatives of MLN-4924, wherein the active derivative retains the ability to inhibit the activity of Perforin-2.

In other embodiments, the compound that inhibits Perforin-2 activity comprises an NAE inhibitor compound referred to herein as cyclometallated rhodium(III) complex [Rh(ppy)₂(dppz)] (complex 1) (where ppy=2-phenylpyridine and dppz=dipyrido[3,2-a:2′,3′-c]phenazine dipyridophenazine) See, Zhong H-J, et al. (2012) PLoS ONE 7(11): e49574; herein incorporated by reference in its entirety. Further provided are active derivatives of rhodium(III) complex [Rh(ppy)₂(dppz)] (complex I), wherein the active derivative retains the ability to inhibit the activity of Perforin-2. Various derivatives of rhodium(III) complex [Rh(ppy)₂(dppz)] are known in the art and comprise complexes 2, 3 and 4. For the various complexes R is defined as: Complex 1: R1, R2, R3=H; Complex 2: R1, R2=CH3, R3=H; Complex 3: R1, R2=CH3, R3=CHO; and Complex 4: R1=H, R2=NO2, R3+CHO. The chemical structure of the cyclometallated rhodium(III) complex [Rh(ppy)₂(dppz)]⁺ is shown in FIG. 27B.

The term “active derivative” refers to a variant of any of the various compounds that modulate Perforin-2 activity provided herein which contain structural modifications and retain the Perforin-2 modulation properties. In the case of a compound that inhibits Perforin-2 activity, an active variant of that compound retains the ability to inhibit Perforin-2 activity. In the case of a compound that increases Perforin-2 activity, an active variant of that compound retains the ability to increase Perorin-2 activity.

In some cases, neddylation can be inactivated by a deamidase. Thus, in some embodiments, a compound that inhibits Perforin-2 activity is a deamidase. In a specific embodiment, the deamidase is Cif. See, for example, Taieb, F, et al. (2011) Toxins (Basel) 3(4):356-68, herein incorporated by reference in its entirety.

In another embodiment, Perforin-2 activity is inhibited by a Cullin Ring Ubiquitin Ligase (CRL) inhibitor. A non-limiting example of a CRL inhibitor is MLN-4924. In a specific embodiment the Cullin Ring Ubiquitin Ligase inhibitor comprises MLN-4924.

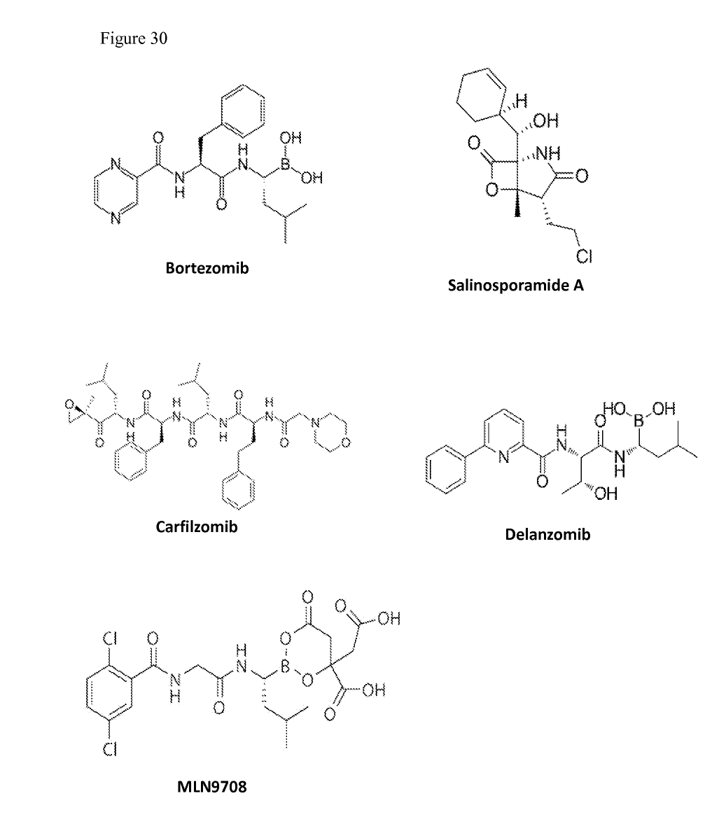

In other embodiments, Perforin-2 activity is inhibited by a proteasome inhibitor. Non-limiting examples of proteasome inhibitors include, for example, Bortezomib, Salinosporamide A, Carfilzomib, MLN9708, Delanzomib (CEP-18770) or active derivatives thereof. The structures of non-limiting examples of proteasome inhibitors are shown in FIG. 30. In a specific embodiment, the proteasome inhibitor comprises Bortezomib, Salinosporamide A, Carfilzomib, MLN9708, Delanzomib or an active derivative thereof.

In non-limiting embodiments, the compound that inhibits Perforin-2 activity can modulate the activity and/or expression of one or more of the following target pathways and/or molecules: any component of the ubiquitination pathway, ubiquitin, E1 ubiquitin-activating enzyme, E2 ubiquitin-conjugating enzyme, E3 ubiquitin ligase, Cullin ring ubiquitin ligase (CRL), any component of the neddylation pathway, NEDD8, NEDD8 activating enzyme (NAE), an isopeptidase, a deubiquitinase, a deamidase, Cif, a deneddylase, Ubc12, βTrcP, Skp1, Cullin1, Vps34, RASA2, Ubc4, Rbx1, proteasome, TEC, NEK9, Mapk12, and/or Perforin-2.

B. Compounds that Increase Perforin-2 Activity

Methods and compositions are also provided that employ compounds which increase Perforin-2 activity. Such compounds find use in, for example, treating a subject suffering from an infectious disease organism.

Provided herein are various components of the molecular pathway responsible for activation of Perforin-2. A key discovery is that ubiquitination of Perforin-2 is an important step in the polymerization and activation of Perforin-2 (see Examples 1-3 provided elsewhere herein). Therefore, any of the various components of the Perforin-2 activation pathway provided herein could be modulated and result in an increase in Perforin-2 activity.

Various compounds which increase the activity of Perforin-2 are provided herein (i.e. compounds that result in the modulation of any one or more of the various components of the Perforin-2 activation pathway). In one embodiment, the compounds which increase the activity of Perforin-2 increase the ubiquitination of Perforin-2.

As used herein, “increase”, “increases” or “increasing” refers to any significant increase in one or more biological activities and/or expression of a target (i.e. a target polypeptide or a target signaling pathway) as compared to an appropriate control. An increase can be any statistically significant increase of at least 5%, 10%, 15%, 20%, 25%, 30%, 40%, 50%, 60%, 70%, 80%, 85%, 90%, 95%, 96%, 97%, 98%, 99%, 100%, 200%, 400% or more as compared to an appropriate control. Alternatively, an increase can be any fold increase of at least 1.5-fold, 2-fold, 3-fold, 4-fold, 5-fold, 6-fold, 7-fold, 8-fold, 9-fold, 10-fold, 12-fold, 14-fold, 16-fold, 20-fold or more as compared to an appropriate control.

An increase in Perforin-2 activity can be assayed in a variety of ways, including, but not limited to, an increase in the level of Perforin-2 protein by protein expression analysis such as Western blot, immunoprecipitation, immunohistochemistry, immunofluorescence, or an increase in Perforin-2 mRNA expression by analysis such as Northern blot or RT-PCR. In addition, an increase in the activity of Perforin-2 can be measured by assaying for an increase in the bactericidal activity of a cell infected with bacteria as compared to an appropriate control. Methods for assaying include, but are not limited to, a decrease in bacterial replication, or a decrease in cell death of the infected cells. An increase in Perforin-2 activity can also be measured in vivo by measuring for a decrease in bacterial colony forming units in various organs and blood after infection with a bacterium as compared to an appropriate control. Various assays to measure Perforin-2 activity are described elsewhere herein.

As used herein, “a compound that increases Perforin-2 activity” refers to a compound that modulates the activity of at least one component of the Perforin-2 activation pathway. In some embodiments the compound that increases Perforin-2 activity increases the activity and/or expression of one or more components of the Perforin-2 activation pathway, thereby increasing Perforin-2 activity. In other embodiments, the compound that increases Perforin-2 activity decreases the activity and/or expression of one or more components of the Perforin-2 activation pathway, thereby increasing Perforin-2 activity.

In some embodiments, the compound that increases Perforin-2 activity increases the ubiquitination of Perforin-2. In specific embodiments, the compound increases the activity and/or expression of at least one component of the ubiquitination pathway. As used herein, a “component of the ubiquitination pathway” refers to any molecule that is involved in the addition and/or removal of ubiquitin on a target molecule. For a review of the ubiquitin pathway, see, for example, Vlachostergios, P J, et al. (2013) Growth Factors 31(3):106-13, which is herein incorporated by reference in its entirety. Components of the ubiquitination pathway can include, for example, ubiquitin, any E1 ubiquitin-activating enzyme, any E2 ubiquitin-conjugating enzyme, any E3 ubiquitin ligase, any component of the neddylation pathway, NEDD8, NEDD8 activating enzyme (NAE), deneddylase, deamidase, Cullin ring ubiquitin ligase (CRL), Ubc12, βTrcP, Skp1, Cullin1, Ubc4, Rbx1, proteasome, isopeptidases or deubiquitinases.

In further embodiments, the at least one component of the ubiquitination pathway comprises an E1 ubiquitin-activating enzyme, an E2 ubiquitin-conjugating enzyme or an E3 ubiquitin ligase.

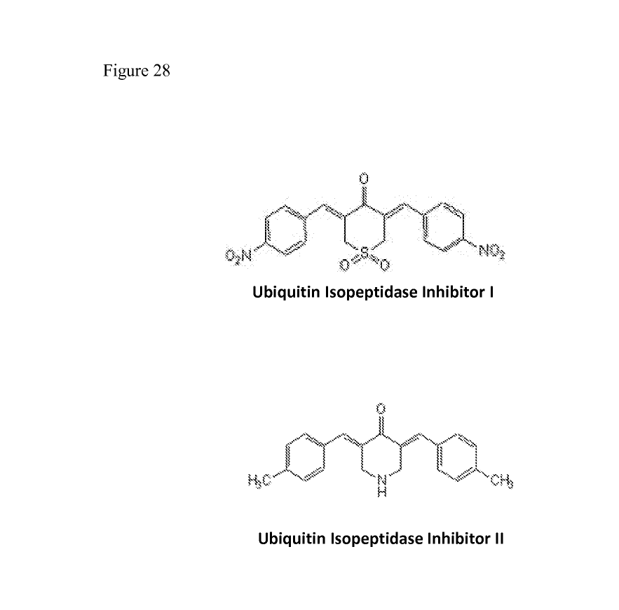

In yet further embodiments, the at least one compound comprises an isopeptidase inhibitor. In specific embodiments, the isopeptidase inhibitor comprises Ubiquitin Isopeptidase Inhibitor II (F6) (3,5-bis((4-Methylphenyl)methylene)-1,1-dioxide, piperidin-4-one) or Ubiquitin Isopeptidase Inhibitor I (G5) (3,5-bis((4-Nitrophenyl)methylene)-1,1-dioxide, tetrahydro-4H-thiopyran-4-one) or active derivatives thereof. The chemical structures for the isopeptidase inhibitors provided herein are depicted in FIG. 28.

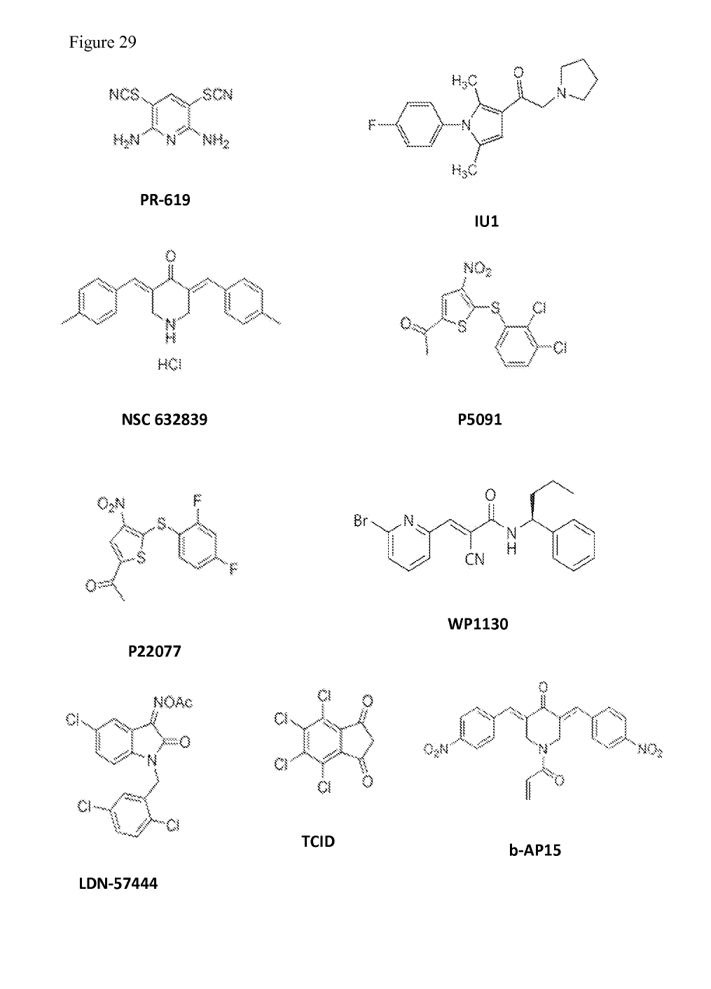

In another embodiment, the at least one compound that increases ubiquitination of Perforin-2 comprises a deubiquitinase inhibitor. In specific embodiments, the deubiquitinase inhibitor comprises PR-619, IU1, NSC 632839, P5091, p22077, WP1130, LDN-57444, TCID, b-AP15 or an active derivative thereof. The chemical structures for the various deubiquitinase inhibitors provided herein are shown in FIG. 29.

Also provided herein, is the finding that neddylation is an important step in the ubiquitination pathway leading to Perforin-2 activation (see Examples 1-3 provided elsewhere herein). As used herein, “neddylation” refers to the conjugation of NEDD8 to a target molecule. In one embodiment, the at least one compound that increases ubiquitination of Perforin-2 modulates the activity and/or expression of at least one component of the neddylation pathway. As used herein, a “component of the neddylation pathway” refers to any molecule involved in the neddylation or deneddylation of a target molecule. By, “deneddylation” is meant the removal and/or deactivation of NEDD8 on a target molecule. For example, NEDD8 can be removed by a deneddylase or deactivated by a deamidase. Non-limiting examples of the components of the neddylation pathway include, for example, NEDD8, NEDD8 activating enzyme (NAE), a deneddylase or a deamidase.

In specific embodiments, the compound that increases Perforin-2 ubiquitination is a deneddylation inhibitor. In a further embodiment, the deneddylation inhibitor comprises PR-619, Ubiquitin Isopeptidase Inhibitor II (F6) (3,5-bis((4-Methylphenyl)methylene)-1,1-dioxide, piperidin-4-one), Ubiquitin Isopeptidase Inhibitor I (G5) (3,5-bis((4-Nitrophenyl)methylene)-1,1-dioxide, tetrahydro-4H-thiopyran-4-one) or active derivatives thereof.

In non-limiting embodiments, the compound that increases Perforin-2 activity can modulate the activity and/or expression of one or more of the following target pathways and/or molecules: any component of the ubiquitination pathway, ubiquitin, E1 ubiquitin-activating enzyme, E2 ubiquitin-conjugating enzyme, E3 ubiquitin ligase, Cullin ring ubiquitin ligase (CRL), any component of the neddylation pathway, an isopeptidase, a deubiquitinase, NEDD8, NEDD8 activating enzyme (NAE), a deamidase, a deneddylase, Ubc12, βTrcP, Skp1, Cullin1, Vps34, RASA2, Ubc4, Rbx1, proteasome, TEC, NEK9, Mapk12, and/or Perforin-2.

C. Various Types of Compounds that Modulate Perforin-2 Activity

The compounds that modulate the Perforin-2 activation pathway comprise a variety of different agents. For example, a compound can comprise small molecules, polypeptides, polynucleotides, oligonucleotides, antibodies, and mediators of RNA interference. Non-limiting examples of such compounds are disclosed below.

In some embodiments, a compound that modulates Perforin-2 activity comprises a small molecule, a polypeptide, an oligonucleotide, a polynucleotide or combinations thereof. In specific embodiments, a compound that inhibits Perforin-2 activity comprises MLN-4924 or an active derivative thereof.

The use of the term “polynucleotide” is not intended to limit the present invention to polynucleotides comprising DNA. Those of ordinary skill in the art will recognize that polynucleotides, can comprise ribonucleotides and combinations of ribonucleotides and deoxyribonucleotides. Such deoxyribonucleotides and ribonucleotides include both naturally occurring molecules and synthetic analogues.

As used herein, the term “oligonucleotide,” is meant to encompass all forms of RNA, DNA, or RNA/DNA molecules.

The polypeptides, polynucleotides and oligonucleotides disclosed herein may be altered in various ways including amino acid substitutions, nucleotide substitutions, deletions, truncations, and insertions. Methods for such manipulations are generally known in the art. For example, amino acid sequence variants and fragments of the components of the Perforin-2 activation pathway can be prepared by mutations in the DNA. Methods for mutagenesis and polynucleotide alterations are well known in the art. See, for example, Kunkel (1985) Proc. Natl. Acad. Sci. USA 82:488-492; Kunkel et al. (1987) Methods in Enzymol. 154:367-382; U.S. Pat. No. 4,873,192; Walker and Gaastra, eds. (1983) Techniques in Molecular Biology (MacMillan Publishing Company, New York) and the references cited therein.

i. Small Molecules

Small molecule test compounds can initially be members of an organic or inorganic chemical library. As used herein, “small molecules” refers to small organic or inorganic molecules of molecular weight below about 3,000 Daltons. The small molecules can be natural products or members of a combinatorial chemistry library. A set of diverse molecules should be used to cover a variety of functions such as charge, aromaticity, hydrogen bonding, flexibility, size, length of side chain, hydrophobicity, and rigidity. Combinatorial techniques suitable for synthesizing small molecules are known in the art, e.g., as exemplified by Obrecht and Villalgordo, Solid-Supported Combinatorial and Parallel Synthesis of Small-Molecular-Weight Compound Libraries, Pergamon-Elsevier Science Limited (1998), and include those such as the “split and pool” or “parallel” synthesis techniques, solid-phase and solution-phase techniques, and encoding techniques (see, for example, Czarnik, Curr. Opin. Chem. Bio., 1:60 (1997). In addition, a number of small molecule libraries are commercially available.

In some embodiments, a compound that modulates Perforin-2 activity comprises a small molecule. In specific embodiments, the small molecule comprises MLN-4924 or an active derivative thereof.

ii. Antibodies

In one embodiment, the modulators of Perforin-2 activity can comprise an antibody. Thus, in specific embodiments, antibodies against the any of the various components of the Perforin-2 activation pathway are provided. Antibodies, can include either polyclonal and/or monoclonal antibodies (mAbs) which can be made by standard protocols. See, for example, Harlow and Lane, Using Antibodies: A Laboratory Manual, CSHL, New York, 1999. Techniques for conferring immunogenicity on a protein or peptide include conjugation to carriers or other techniques are also known in the art. In preferred embodiments, the subject antibodies are immunospecific for the unique antigenic determinants of any polypeptide of any of the various components of the Perforin-2 activation pathway, including but not limited to, any component of the ubiquitination pathway, ubiquitin, E1 ubiquitin-activating enzyme, E2 ubiquitin-conjugating enzyme, E3 ubiquitin ligase, Cullin ring ubiquitin ligase (CRL), any component of the neddylation pathway, an isopeptidase, a deubiquitinase, NEDD8, NEDD8 activating enzyme (NAE), a deamidase, a deneddylase, Ubc12, βTrcP, Skp1, Cullin1, Vps34, RASA2, Ubc4, Rbx1, proteasome, TEC, NEK9, Mapk12, and/or Perforin-2.

As discussed herein, these antibodies are collectively referred to as “anti-Perforin-2 activation pathway antibodies” and can include antagonistic antibodies that block activity of a component of the Perforin-2 activation pathway or antibodies that promote activity of a component of the Perforin-2 activation pathway. The antibodies can be used alone or in combination in the methods of the invention.

By “antibodies that specifically bind” is intended that the antibodies will not substantially cross react with another polypeptide. By “not substantially cross react” is intended that the antibody or fragment has a binding affinity for a non-homologous protein which is less than 10%, less than 5%, or less than 1%, of the binding affinity for the target protein.

The various modulating antibodies disclosed herein and for use in the methods of the present invention can be produced using any antibody production method known to those of skill in the art. Thus, the modulating antibodies can be polyclonal or monoclonal.

By “monoclonal antibody” is intended an antibody obtained from a population of substantially homogeneous antibodies, that is, the individual antibodies comprising the population are identical except for possible naturally occurring mutations that may be present in minor amounts.

By “epitope” is intended the part of an antigenic molecule to which an antibody is produced and to which the antibody will bind. Epitopes can comprise linear amino acid residues (i.e., residues within the epitope are arranged sequentially one after another in a linear fashion), nonlinear amino acid residues (referred to herein as “nonlinear epitopes”—these epitopes are not arranged sequentially), or both linear and nonlinear amino acid residues.

Additionally, the term “antibody” as used herein encompasses chimeric and humanized anti-Perforoin-2 activation pathway antibodies. By “chimeric” antibodies is intended antibodies that are most preferably derived using recombinant deoxyribonucleic acid techniques and which comprise both human (including immunologically “related” species, e.g., chimpanzee) and non-human components. Thus, the constant region of the chimeric antibody is most preferably substantially identical to the constant region of a natural human antibody; the variable region of the chimeric antibody is most preferably derived from a non-human source and has the desired antigenic specificity to a polypeptide of the Perforin-2 activation pathway. The non-human source can be any vertebrate source that can be used to generate antibodies to a polypeptide of the Perforin-2 activation pathway or material comprising a polypeptide of the Perforin-2 activation pathway. Such non-human sources include, but are not limited to, rodents (e.g., rabbit, rat, mouse, etc.; see, e.g., U.S. Pat. No. 4,816,567) and non-human primates (e.g., Old World Monkeys, Apes, etc.; see, e.g., U.S. Pat. Nos. 5,750,105 and 5,756,096).

By “humanized” is intended forms of anti-Perforin-2 activation pathway antibodies that contain minimal sequence derived from non-human immunoglobulin sequences. Accordingly, such “humanized” antibodies may include antibodies wherein substantially less than an intact human variable domain has been substituted by the corresponding sequence from a non-human species.

iii. Silencing Elements

The compound that modulates Perforin-2 activity can further comprise a silencing element which targets a sequence of any one of the components of the Perforin-2 activation pathway and thereby modulates the activity of Perforin-2. Such silencing elements can be designed to target a variety of sequences, including any sequence encoding a polypeptide in the Perforin-2 activation pathway including, for example, the sequences encoding the polypeptides of any component of the ubiquitination pathway, ubiquitin, E1 ubiquitin-activating enzyme, E2 ubiquitin-conjugating enzyme, E3 ubiquitin ligase, Cullin ring ubiquitin ligase (CRL), any component of the neddylation pathway, an isopeptidase, a deubiquitinase, NEDD8, NEDD8 activating enzyme (NAE), a deamidase, a deneddylase, Ubc12, βTrcP, Skp1, Cullin1, Vps34, RASA2, Ubc4, Rbx1, proteasome, TEC, NEK9, Mapk12, and/or Perforin-2.

By “silencing element” is intended a polynucleotide which when expressed or introduced into a host cell is capable of reducing or eliminating the level or expression of a target polynucleotide or the polypeptide encoded thereby. The silencing element employed can reduce or eliminate the expression level of the target sequence by influencing the level of the target RNA transcript or, alternatively, by influencing translation and thereby affecting the level of the encoded polypeptide. Methods to assay for functional silencing elements that are capable of reducing or eliminating the level of a sequence of interest are disclosed elsewhere herein. Silencing elements can include, but are not limited to, a sense suppression element, an antisense suppression element, a siRNA, a shRNA, a protein nucleic acid (PNA) molecule, a miRNA, a hairpin suppression element, or any precursor thereof.

Thus, a silencing element can comprise a template for the transcription of a sense suppression element, an antisense suppression element, a siRNA, a shRNA, a miRNA, or a hairpin suppression element; an RNA precursor of an antisense RNA, a siRNA, an shRNA, a miRNA, or a hairpin RNA; or, an active antisense RNA, siRNA, shRNA, miRNA, or hairpin RNA. Methods of introducing the silencing element into the cell may vary depending on which form (DNA template, RNA precursor, or active RNA) is introduced into the cell. When the silencing element comprises a DNA molecule encoding an antisense suppression element, a siRNA, a shRNA, a miRNA, or a hairpin suppression element an interfering RNA, it is recognized that the DNA can be designed so that it is transiently present in a cell or stably incorporated into the genome of the cell. Such methods are discussed in further detail elsewhere herein.

The silencing element can reduce or eliminate the expression level of a target sequence by influencing the level of the target RNA transcript, by influencing translation and thereby affecting the level of the encoded polypeptide, or by influencing expression at the pre-transcriptional level (i.e., via the modulation of chromatin structure, methylation pattern, etc., to alter gene expression). See, for example, Verdel et al. (2004) Science 303:672-676; Pal-Bhadra et al. (2004) Science 303:669-672; Allshire (2002) Science 297:1818-1819; Volpe et al. (2002) Science 297:1833-1837; Jenuwein (2002) Science 297:2215-2218; and Hall et al. (2002) Science 297:2232-2237. Methods to assay for functional interfering RNA that are capable of reducing or eliminating the level of a sequence of interest are disclosed elsewhere herein.

As used herein, a “target sequence” comprises any sequence that one desires to decrease the level of expression. By “reducing the expression level of a polynucleotide or a polypeptide encoded thereby” is intended to mean, the polynucleotide or polypeptide level of the target sequence is statistically lower than the polynucleotide level or polypeptide level of the same target sequence in an appropriate control which is not exposed to the silencing element. In particular embodiments, reducing the polynucleotide level and/or the polypeptide level of the target sequence according to the presently disclosed subject matter results in less than 95%, less than 90%, less than 80%, less than 70%, less than 60%, less than 50%, less than 40%, less than 30%, less than 20%, less than 10%, or less than 5% of the polynucleotide level, or the level of the polypeptide encoded thereby, of the same target sequence in an appropriate control. Methods to assay for the level of the RNA transcript, the level of the encoded polypeptide, or the activity of the polynucleotide or polypeptide are discussed elsewhere herein.

Any region or multiple regions of a target polynucleotide can be used to design a domain of the silencing element that shares sufficient sequence identity to allow the silencing element to decrease the level of the target polynucleotide. For instance, the silencing element can be designed to share sequence identity to the 5′ untranslated region of the target polynucleotide(s), the 3′ untranslated region of the target polynucleotide(s), exonic regions of the target polynucleotide(s), intronic regions of the target polynucleotide(s), and any combination thereof.

The ability of a silencing element to reduce the level of the target polynucleotide may be assessed directly by measuring the amount of the target transcript using, for example, Northern blots, nuclease protection assays, reverse transcription (RT)-PCR, real-time RT-PCR, microarray analysis, and the like. Alternatively, the ability of the silencing element to reduce the level of the target polynucleotide may be measured directly using a variety of affinity-based approaches (e.g., using a ligand or antibody that specifically binds to the target polypeptide) including, but not limited to, Western blots, immunoassays, ELISA, flow cytometry, protein microarrays, and the like. In still other methods, the ability of the silencing element to reduce the level of the target polynucleotide can be assessed indirectly, e.g., by measuring a functional activity of the polypeptide encoded by the transcript or by measuring a signal produced by the polypeptide encoded by the transcript.

D. Kits

As used herein, “kit” comprises a modulator of Perforin-2 as described herein for use in modulating the activity of Perforin-2 in biological samples. The terms “kit” and “system,” as used herein are intended to refer to at least one or more compound that modulates Perforin-2 activity which, in specific embodiments, are in combination with one or more other types of elements or components (e.g., other types of biochemical reagents, containers, packages, such as packaging intended for commercial sale, substrates to which detection reagents are attached, electronic hardware components, instructions of use, and the like).

In some embodiments, the kit comprises the compound MLN-4924 or an active derivative thereof.

III. Uses and Methods

The various components of the Perforin-2 activation pathway and the various compounds that modulate Perforin-2 activity disclosed herein can be used in various methods including screening assays, diagnostic and prognostic assays, methods of modulating Perforin-2 activity and methods of treatment (e.g., therapeutic and prophylactic).

A. Methods for Modulating the Activity of the Perforin-2 Pathway

Methods for modulating the activity of Perforin-2 in a subject are provided. Such methods comprise administering at least one modulator of Perforin-2 activity to a subject in need thereof. Any of the various components of the Perforin-2 activation pathway disclosed herein can be modulated by the methods provided herein.

The various compounds that inhibit Perforin-2 activity find use in treating any conditions associated with gut inflammation. For example, Perforin-2 inhibitors find use in treating colitis, ulcerative colitis, Crohn's disease or inflammatory bowel disease. Thus, in one embodiment, a method of treating a subject having inflammation of the gut is provided. Such a method comprises administering to the subject a therapeutically effective amount of at least one compound that inhibits Perforin-2 activity. The compounds can modulate any of the various components of the Perforin-2 activation pathway disclosed herein. Various compounds that inhibit Perforin-2 activity are discussed elsewhere herein.

In specific embodiments, the method can employ a compound that inhibits Perforin-2 activity that is a small molecule, such as the small molecule MLN-4924 or an active derivative thereof.

A method of treating a subject suffering from an infectious disease organism is provided herein. Such a method comprises administering to the subject a therapeutically effective amount of at least one compound that increases Perforin-2 activity. The compounds that increase Perforin-2 activity can modulate any of the various components of the Perforin-2 activation pathway disclosed herein. Various compounds that increase Perforin-2 activity are discussed elsewhere herein. In specific embodiments, the compound increases the ubiquitination of Perforin-2.

A method of increasing Perforin-2 activity is provided. Such a method comprises administering to a subject in need thereof, a therapeutically effective amount of at least one compound that increases the ubiquitination of Perforin-2 and thereby increases the activity of Perforin-2. Any of the various components of the ubiquitination pathway disclosed herein can be modulated by any of the various compounds that modulate Perforin-2 activity provided herein. In one embodiment, the compound increases the activity and/or expression of at least one component of the ubiquitination pathway.

A therapeutically effective amount of a modulator of Perforin-2 activity can be administered to a subject. By “therapeutically effective amount” is intended an amount that is useful in the treatment, prevention or diagnosis of a disease or condition. As used herein, a therapeutically effective amount of a Perforin-2 modulator is an amount which, when administered to a subject, is sufficient to achieve a desired effect, such as, for example in the case of an inhibitor, decreasing Perforin-2 activity in a subject being treated with that composition without causing a substantial cytotoxic effect in the subject. A therapeutically effective amount for treating gut inflammation will result in a decrease in gut inflammation. A decrease in gut inflammation can be measured, for example, by a decrease in symptoms and/or indicators of gut inflammation. For example, a decrease in gut inflammation can be detected by measuring inflammatory markers in the stool or by a colonoscopy and/or biopsy of the pathological lesions. For the case of an activator of Perforin-2, the desired effect to be achieved would be, for example, increasing Perforin-2 activity in a subject being treated with that composition without causing a substantial cytotoxic effect in the subject. The effective amount of a Perforin-2 modulator useful for modulating Perforin-2 activity will depend on the subject being treated, the severity of the affliction, and the manner of administration of the Perforin-2 inhibitor.

By “subject” is intended mammals, e.g., primates, humans, agricultural and domesticated animals such as, but not limited to, dogs, cats, cattle, horses, pigs, sheep, and the like. Preferably the subject undergoing treatment with the pharmaceutical formulations of the invention is a human.

When administration is for the purpose of treatment, administration may be for either a prophylactic or therapeutic purpose. When provided prophylactically, the substance is provided in advance of any symptom. The prophylactic administration of the substance serves to prevent or attenuate any subsequent symptom. When provided therapeutically, the substance is provided at (or shortly after) the onset of a symptom. The therapeutic administration of the substance serves to attenuate any actual symptom.

The skilled artisan will appreciate that certain factors may influence the dosage required to effectively treat a subject, including but not limited to the severity of the disease or disorder, previous treatments, the general health and/or age of the subject, and other diseases present. Moreover, treatment of a subject with a therapeutically effective amount of a modulator of Perforin-2 activity (including an inhibitor such as MLN-4924) can include a single treatment or, preferably, can include a series of treatments. It will also be appreciated that the effective dosage of a modulator of Perforin-2 activity used for treatment may increase or decrease over the course of a particular treatment.

It is understood that appropriate doses of such active compounds depends upon a number of factors within the knowledge of the ordinarily skilled physician, veterinarian, or researcher. The dose(s) of the active compounds will vary, for example, depending upon the identity, size, and condition of the subject or sample being treated, further depending upon the route by which the composition is to be administered, if applicable, and the effect which the practitioner desires the active compound to have upon the Perforin-2 activation pathway. Exemplary doses include milligram or microgram amounts of the small molecule per kilogram of subject or sample weight (e.g., about 1 microgram per kilogram to about 500 milligrams per kilogram, about 100 micrograms per kilogram to about 5 milligrams per kilogram, or about 1 microgram per kilogram to about 50 micrograms per kilogram. It is furthermore understood that appropriate doses of an active agent depend upon the potency of the active agent with respect to the expression or activity to be modulated. Such appropriate doses may be determined using the assays described herein. When one or more of these small molecules is to be administered to an animal (e.g., a human) in order to modulate activity of Perforin-2, a physician, veterinarian, or researcher may, for example, prescribe a relatively low dose at first, subsequently increasing the dose until an appropriate response is obtained. In addition, it is understood that the specific dose level for any particular animal subject will depend upon a variety of factors including the activity of the specific compound employed, the age, body weight, general health, gender, and diet of the subject, the time of administration, the route of administration, the rate of excretion, any drug combination, and the degree of expression or activity to be modulated.

Therapeutically effective amounts of a modulator of Perforin-2 activity can be determined by animal studies. When animal assays are used, a dosage is administered to provide a target tissue concentration similar to that which has been shown to be effective in the animal assays. It is recognized that the method of treatment may comprise a single administration of a therapeutically effective amount or multiple administrations of a therapeutically effective amount of the modulator of Perforin-2 activity.

In specific embodiments, the therapeutically effective amount of MLN-4924 is between 50 μg/kg and 100 mg/kg. For example, the daily dosage amount can be for example about 50, about 100, about 150, about 200, about 250, about 300, about 350, about 400, about 450, about 500, about 600, about 700, about 800, or about 900 μg/kg. Additionally, the daily dosage amount can be for example about 1, about 2, about 3, about 4, about 5, about 6, about 7, about 8, about 9, about 10, about 15, about 20, about 25, about 30, about 35, about 40, about 45, about 50, about 55, about 60, about 65, about 70, about 75, about 80, about 85, about 90, about 95, or about 100 mg/kg.

i. Infectious Organisms

As used herein, “infectious organisms” or “infectious disease organisms” can include, but are not limited to, for example, bacteria, viruses, fungi, parasites and protozoa.

Various infectious organisms are encompassed by the methods and compositions provided herein. In some embodiments, the compound that modulates Perforin-2 activity inhibits replication, inhibits growth, or induces death of an infectious disease organism. In specific embodiments, the infectious disease organism is an intracellular or extracellular bacterium.

Non-limiting examples of the various infectious disease organisms encompassed by the methods and compositions provided herein include:

Particularly preferred bacteria causing serious human diseases are the Gram positive organisms: Staphylococcus aureus, Methicillin-resistant Staphylococcus aureus (MRSA), Staphylococcus epidermidis, Enterococcus faecalis and E. faecium, Streptococcus pneumoniae and the Gram negative organisms: Pseudomonas aeruginosa, Burkholdia cepacia, Xanthomonas maltophila, Escherichia coli, Enteropathogenic E. coli (EPEC), Enterobacter spp, Klebsiella pneumonia, Chlamydia spp., including Chlamydia trachomatis, and Salmonella spp., including Salmonella typhimurium.

In another preferred embodiment, the bacteria are Gram negative bacteria. Examples, comprise: Pseudomonas aeruginosa; Burkholdia cepacia; Xanthomonas maltophila; Escherichia coli; Enterobacter spp.; Klebsiella pneumoniae; Salmonella spp.

The present invention also provides methods for treating diseases include infections by Mycobacterium spp., Mycobacterium tuberculosis, Mycobacterium smegmatis, Mycobacterium avium, Yersinia pseudotuberculosis, Entamoeba histolytica; Pneumocystis carinii, Trypanosoma cruzi, Trypanosoma brucei, Leishmania mexicana, Listeria monocytogenes, Shigella flexneri, Clostridium histolyticum, Staphylococcus aureus, foot-and-mouth disease virus and Crithidia fasciculata; as well as in osteoporosis, autoimmunity, schistosomiasis, malaria, tumor metastasis, metachromatic leukodystrophy, muscular dystrophy and amytrophy.

Other examples include veterinary and human pathogenic protozoa, intracellular active parasites of the phylum Apicomplexa or Sarcomastigophora, Trypanosoma, Plasmodia, Leishmania, Babesia and Theileria, Cryptosporidia, Sacrocystida, Amoeba, Coccidia and Trichomonadia. These compounds are also suitable for the treatment of Malaria tropica, caused by, for example, Plasmodium falciparum, Malaria tertiana, caused by Plasmodium vivax or Plasmodium ovale and for the treatment of Malaria quartana, caused by Plasmodium malariae. They are also suitable for the treatment of Toxoplasmosis, caused by Toxoplasma gondii, Coccidiosis, caused for instance by Isospora belli, intestinal Sarcosporidiosis, caused by Sarcocystis suihominis, dysentery caused by Entamoeba histolytica, Cryptosporidiosis, caused by Cryptosporidium parvum, Chagas' disease, caused by Trypanosoma cruzi, sleeping sickness, caused by Trypanosoma brucei rhodesiense or gambiense, the cutaneous and visceral as well as other forms of Leishmaniosis. They are also suitable for the treatment of animals infected by veterinary pathogenic protozoa, like Theileria parva, the pathogen causing bovine East coast fever, Trypanosoma congolense congolense or Trypanosoma vivax vivax, Trypanosoma brucei brucei, pathogens causing Nagana cattle disease in Africa, Trypanosoma brucei evansi causing Surra, Babesia bigemina, the pathogen causing Texas fever in cattle and buffalos, Babesia bovis, the pathogen causing European bovine Babesiosis as well as Babesiosis in dogs, cats and sheep, Sarcocystis ovicanis and ovifelis pathogens causing Sarcocystiosis in sheep, cattle and pigs, Cryptosporidia, pathogens causing Cryptosporidioses in cattle and birds, Eimeria and Isospora species, pathogens causing Coccidiosis in rabbits, cattle, sheep, goats, pigs and birds, especially in chickens and turkeys. Rickettsia comprise species such as Rickettsia felis, Rickettsia prowazekii, Rickettsia rickettsii, Rickettsia typhi, Rickettsia conorii, Rickettsia africae and cause diseases such as typhus, rickettsialpox, Boutonneuse fever, African Tick Bite Fever, Rocky Mountain spotted fever, Australian Tick Typhus, Flinders Island Spotted Fever and Queensland Tick Typhus. In the treatment of these diseases, the compounds of the present invention may be combined with other agents.

Particularly preferred fungi causing or associated with human diseases according to the present invention include (but not restricted to) Candida albicans, Histoplasma neoformans, Coccidioides immitis and Penicillium marneffei.

B. Pharmaceutical Compositions

The compounds that modulate Perforin-2 activity disclosed herein can be incorporated into pharmaceutical compositions suitable for administration. Such compositions typically comprise one or more compounds that modulate Perforin-2 activity and a pharmaceutically acceptable carrier. In specific embodiments, the pharmaceutical composition comprises MLN-4924 or an active derivative thereof.

As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

The pharmaceutical compositions of the invention may contain, for example, more than one agent which may act independently of the other on a different target molecule. In some examples, a pharmaceutical composition of the invention, containing one or more compounds of the invention, is administered in combination with another useful composition such as an anti-inflammatory agent, an immunostimulator, a chemotherapeutic agent, an antibacterial agent, or the like. Furthermore, the compositions of the invention may be administered in combination with a cytotoxic, cytostatic, or chemotherapeutic agent such as an alkylating agent, anti-metabolite, mitotic inhibitor or cytotoxic antibiotic, as described above. In general, the currently available dosage forms of the known therapeutic agents for use in such combinations will be suitable.

Combination therapy (or “co-therapy”) includes the administration of a therapeutic composition and at least a second agent as part of a specific treatment regimen intended to provide the beneficial effect from the co-action of these therapeutic agents. The beneficial effect of the combination includes, but is not limited to, pharmacokinetic or pharmacodynamic coactions resulting from the combination of therapeutic agents. Administration of these therapeutic agents in combination typically is carried out over a defined time period (usually minutes, hours, days or weeks depending upon the combination selected).

Combination therapy may, but generally is not, intended to encompass the administration of two or more of these therapeutic agents as part of separate monotherapy regimens that incidentally and arbitrarily result in the combinations of the present invention. Combination therapy is intended to embrace administration of these therapeutic agents in a sequential manner, that is, wherein each therapeutic agent is administered at a different time, as well as administration of these therapeutic agents, or at least two of the therapeutic agents, in a substantially simultaneous manner. Substantially simultaneous administration can be accomplished, for example, by administering to the subject a single capsule having a fixed ratio of each therapeutic agent or in multiple, single capsules for each of the therapeutic agents. Sequential or substantially simultaneous administration of each therapeutic agent can be effected by any appropriate route including, but not limited to, topical routes, oral routes, intravenous routes, intramuscular routes, and direct absorption through mucous membrane tissues. The therapeutic agents can be administered by the same route or by different routes. For example, a first therapeutic agent of the combination selected may be administered by injection while the other therapeutic agents of the combination may be administered topically.

A pharmaceutical composition of the invention is formulated to be compatible with its intended route of administration. Examples of routes of administration include parenteral, e.g., intravenous, intradermal, subcutaneous, oral (e.g., inhalation), transdermal (topical), and transmucosal. In addition, it may be desirable to administer a therapeutically effective amount of the pharmaceutical composition locally to an area in need of treatment. This can be achieved by, for example, local or regional infusion or perfusion during surgery, topical application, injection, catheter, suppository, or implant (for example, implants formed from porous, non-porous, or gelatinous materials, including membranes, such as sialastic membranes or fibers), and the like. In one embodiment, administration can be by direct injection at the site (or former site) of an infection that is to be treated. In another embodiment, the therapeutically effective amount of the pharmaceutical composition is delivered in a vesicle, such as liposomes (see, e.g., Langer, Science 249:1527-33, 1990 and Treat et al., in Liposomes in the Therapy of Infectious Disease and Cancer, Lopez Berestein and Fidler (eds.), Liss, N.Y., pp. 353-65, 1989).

A subject in whom administration of an active component as set forth above is an effective therapeutic regimen for an infection by an infectious disease organism or for inflammation of the gut is preferably a human, but can be any animal. Thus, as can be readily appreciated by one of ordinary skill in the art, the methods and pharmaceutical compositions provided herein are particularly suited to administration to any animal, particularly a mammal, and including, but by no means limited to, domestic animals, such as feline or canine subjects, farm animals, such as but not limited to bovine, equine, caprine, ovine, and porcine subjects, wild animals (whether in the wild or in a zoological garden), research animals, such as mice, rats, rabbits, goats, sheep, pigs, dogs, cats, etc., i.e., for veterinary medical use.

In yet another embodiment, the therapeutically effective amount of the pharmaceutical composition can be delivered in a controlled release system. In one example, a pump can be used (see, e.g., Langer, Science 249:1527-33, 1990; Sefton, Crit. Rev. Biomed. Eng. 14:201-40, 1987; Buchwald et al., Surgery 88:507-16, 1980; Saudek et al., N. Engl. J. Med. 321:574-79, 1989). In another example, polymeric materials can be used (see, e.g., Levy et al., Science 228:190-92, 1985; During et al., Ann. Neurol. 25:351-56, 1989; Howard et al., J. Neurosurg. 71:105-12, 1989). Other controlled release systems, such as those discussed by Langer (Science 249:1527-33, 1990), can also be used.

Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes, or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersions. For intravenous administration, suitable carriers include physiological saline, bacteriostatic water, Cremophor EL [symbol] (BASF; Parsippany, N.J.), or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion, and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride, in the composition. Prolonged absorption of the injectable compositions can be brought about by including in the composition an agent that delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying, which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth, or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring. For administration by inhalation, the compounds are delivered in the form of an aerosol spray from a pressurized container or dispenser that contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art. The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated with each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

In one embodiment, the method comprises the use of viruses for administering any of the various compounds for modulating Perforin-2 activity provided herein or any of the various components of the Perforin-2 activation pathway provided herein to a subject. Administration can be by the use of viruses that express any of the target molecules or agents provided herein, such as recombinant retroviruses, recombinant adeno-associated viruses, recombinant adenoviruses, and recombinant Herpes simplex viruses (see, for example, Mulligan, Science 260:926 (1993), Rosenberg et al., Science 242:1575 (1988), LaSalle et al., Science 259:988 (1993), Wolff et al., Science 247:1465 (1990), Breakfield and Deluca, The New Biologist 3:203 (1991)).

A gene encoding any of the various target molecules or agents provided herein can be delivered using recombinant viral vectors, including for example, adenoviral vectors (e.g., Kass-Eisler et al., Proc. Nat'l Acad. Sci. USA 90:11498 (1993), Kolls et al., Proc. Nat'l Acad. Sci. USA 91:215 (1994), Li et al., Hum. Gene Ther. 4:403 (1993), Vincent et al., Nat. Genet. 5:130 (1993), and Zabner et al., Cell 75:207 (1993)), adenovirus-associated viral vectors (Flotte et al., Proc. Nat'l Acad. Sci. USA 90:10613 (1993)), alphaviruses such as Semliki Forest Virus and Sindbis Virus (Hertz and Huang, J. Vir. 66:857 (1992), Raju and Huang, J. Vir. 65:2501 (1991), and Xiong et al., Science 243:1188 (1989)), herpes viral vectors (e.g., U.S. Pat. Nos. 4,769,331, 4,859,587, 5,288,641 and 5,328,688), parvovirus vectors (Koering et al., Hum. Gene Therap. 5:457 (1994)), pox virus vectors (Ozaki et al., Biochem. Biophys. Res. Comm. 193:653 (1993), Panicali and Paoletti, Proc. Nat'l Acad. Sci. USA 79:4927 (1982)), pox viruses, such as canary pox virus or vaccinia virus (Fisher-Hoch et al., Proc. Nat'l Acad. Sci. USA 86:317 (1989), and Flexner et al., Ann. N.Y. Acad. Sci. 569:86 (1989)), and retroviruses (e.g., Baba et al., J. Neurosurg 79:729 (1993), Ram et al., Cancer Res. 53:83 (1993), Takamiya et al., J. Neurosci. Res 33:493 (1992), Vile and Hart, Cancer Res. 53:962 (1993), Vile and Hart, Cancer Res. 53:3860 (1993), and Anderson et al., U.S. Pat. No. 5,399,346). Within various embodiments, either the viral vector itself, or a viral particle, which contains the viral vector may be utilized in the methods described below.

As an illustration of one system, adenovirus, a double-stranded DNA virus, is a well-characterized gene transfer vector for delivery of a heterologous nucleic acid molecule (for a review, see Becker et al., Meth. Cell Biol. 43:161 (1994); Douglas and Curiel, Science & Medicine 4:44 (1997)). The adenovirus system offers several advantages including: (i) the ability to accommodate relatively large DNA inserts, (ii) the ability to be grown to high-titer, (iii) the ability to infect a broad range of mammalian cell types, and (iv) the ability to be used with many different promoters including ubiquitous, tissue specific, and regulatable promoters. In addition, adenoviruses can be administered by intravenous injection, because the viruses are stable in the bloodstream.

Using adenovirus vectors where portions of the adenovirus genome are deleted, inserts are incorporated into the viral DNA by direct ligation or by homologous recombination with a co-transfected plasmid. In an exemplary system, the essential E1 gene is deleted from the viral vector, and the virus will not replicate unless the E1 gene is provided by the host cell. When intravenously administered to intact animals, adenovirus primarily targets the liver. Although an adenoviral delivery system with an E1 gene deletion cannot replicate in the host cells, the host's tissue will express and process an encoded heterologous protein. Host cells will also secrete the heterologous protein if the corresponding gene includes a secretory signal sequence. Secreted proteins will enter the circulation from tissue that expresses the heterologous gene (e.g., the highly vascularized liver).

Moreover, adenoviral vectors containing various deletions of viral genes can be used to reduce or eliminate immune responses to the vector. Such adenoviruses are E1-deleted, and in addition, contain deletions of E2A or E4 (Lusky et al., J. Virol. 72:2022 (1998); Raper et al., Human Gene Therapy 9:671 (1998)). The deletion of E2b has also been reported to reduce immune responses (Amalfitano et al., J. Virol. 72:926 (1998)). By deleting the entire adenovirus genome, very large inserts of heterologous DNA can be accommodated. Generation of so called “gutless” adenoviruses, where all viral genes are deleted, are particularly advantageous for insertion of large inserts of heterologous DNA (for a review, see Yeh. and Perricaudet, FASEB J. 11:615 (1997)).

High titer stocks of recombinant viruses capable of expressing a therapeutic gene can be obtained from infected mammalian cells using standard methods. For example, recombinant herpes simplex virus can be prepared in Vero cells, as described by Brandt et al., J. Gen. Virol. 72:2043 (1991), Herold et al., J. Gen. Virol. 75:1211 (1994), Visalli and Brandt, Virology 185:419 (1991), Grau et al., Invest. Ophthalmol. Vis. Sci. 30:2474 (1989), Brandt et al., J. Virol. Meth. 36:209 (1992), and by Brown and MacLean (eds.), HSV Virus Protocols (Humana Press 1997).

When the subject treated with a recombinant virus is a human, then the therapy is preferably somatic cell gene therapy. That is, the preferred treatment of a human with a recombinant virus does not entail introducing into cells a nucleic acid molecule that can form part of a human germ line and be passed onto successive generations (i.e., human germ line gene therapy).

The pharmaceutical compositions can be included in a container, pack, or dispenser together with instructions for administration.

C. Methods of Identifying, Classifying, and/or Prognosis and/or Predisposition to Disease States

In some embodiments, a modulation of Perforin-2 activity in a biological sample allows for the identification, classification and/or the prognosis and/or predisposition of the biological sample to a disease state or the likelihood of a therapeutic response to a modulator of Perforin-2. More particularly, an increase in Perforin-2 activity allows for the identification, classification and/or the prognosis and/or predisposition of the biological sample to diseases associated with gut inflammation. Various methods and compositions to carry out such methods are disclosed elsewhere herein.

In some embodiments, a method is provided for assaying a biological sample from a subject for an increase in Perforin-2 activity. The method comprises: a) providing a biological sample from the subject; and, b) determining if the biological sample comprises an increase in Perforin-2 activity when compared to an appropriate control. The presence of the increase in Perforin-2 activity when compared to an appropriate control is indicative of a disease associated with gut inflammation. In such a method, the presence of an increase in Perforin-2 activity is indicative of a disease associated with gut inflammation, more particularly, gut inflammation that is responsive to a compound that inhibits Perforin-2 activity. In some embodiments, the disease associated with gut inflammation is, colitis, ulcerative colitis, Crohn's disease or inflammatory bowel disease.

In other embodiments, the increase in Perforin-2 activity comprises a modulation in the activity of a component of the Perforin-2 activation pathway. The component of the Perforin-2 activation pathway can comprise any component of the ubiquitination pathway, ubiquitin, E1 ubiquitin-activating enzyme, E2 ubiquitin-conjugating enzyme, E3 ubiquitin ligase, Cullin ring ubiquitin ligase (CRL), any component of the neddylation pathway, an isopeptidase, a deubiquitinase, NEDD8, NEDD8 activating enzyme (NAE), a deamidase, a deneddylase, Ubc12, βTrcP, Skp1, Cullin1, Vps34, RASA2, Ubc4, Rbx1, proteasome, TEC, NEK9, Mapk12, and/or Perforin-2.

In some embodiments, the biological sample is from the digestive tract, gastrointestinal tract, intestines, lymph nodes, spleen, bone marrow, blood, or the site of inflammation.

In some embodiments, the inhibitor of Perforin-2 activity can be any of the compounds disclosed herein or active derivatives thereof. In specific embodiments, the compound that inhibits Perforin-2 activity comprises MLN-4924 or an active derivative thereof.

D. Methods to Screen for Perforin-2 Pathway Modulating Compounds

Methods are provided for identifying modulating compounds of the Perforin-2 activation pathway (also referred to herein as a “screening assay”). The various components of the Perforin-2 activation pathway provided herein can be used in various assays to screen for Perforin-2 modulating compounds.

In one embodiment, a method of screening for a Perforin-2 inhibitor is provided. Such a method comprises contacting a cell expressing Perforin-2 with a candidate compound, comparing to an appropriate control cell and determining if the candidate compound decreases the activity of Perforin-2.

In another embodiment, a method of screening for a compound that activates Perforin-2 is provided. Such a method comprises contacting a cell expressing Perforin-2 with a candidate compound, comparing to an appropriate control cell and determining if the candidate compound increases the activity of Perforin-2. In specific embodiments, the compound increases the ubiquitination of Perforin-2.

The candidate compounds employed in the various screening assays can include any candidate compound including, for example, polypeptides, peptides, polynucleotides, oligonucleotides, peptidomimetics, small molecules, antibodies, siRNAs, miRNAs, shRNAs, or other drugs. Such candidate compounds can be obtained using any of the numerous approaches in combinatorial library methods known in the art, including biological libraries, spatially addressable parallel solid phase or solution phase libraries, synthetic library methods requiring deconvolution, the “one-bead one-compound” library method, and synthetic library methods using affinity chromatography selection. The biological library approach is limited to peptide libraries, while the other four approaches are applicable to peptide, nonpeptide oligomer, or small molecule libraries of compounds (Lam (1997) Anticancer Drug Des. 12:145).

Examples of methods for the synthesis of molecular libraries can be found in the art, for example in: DeWitt et al. (1993) Proc. Natl. Acad. Sci. USA 90:6909; Erb et al. (1994) Proc. Natl. Acad. Sci. USA 91:11422; Zuckermann et al. (1994). J. Med. Chem. 37:2678; Cho et al. (1993) Science 261:1303; Carrell et al. (1994) Angew. Chem. Int. Ed. Engl. 33:2059; Carell et al. (1994) Angew. Chem. Int. Ed. Engl. 33:2061; and Gallop et al. (1994) J. Med. Chem. 37:1233.

Libraries of compounds may be presented in solution (e.g., Houghten (1992) Bio/Techniques 13:412-421), or on beads (Lam (1991) Nature 354:82-84), chips (Fodor (1993) Nature 364:555-556), bacteria (U.S. Pat. No. 5,223,409), spores (U.S. Pat. Nos. 5,571,698; 5,403,484; and 5,223,409), plasmids (Cull et al. (1992) Proc. Natl. Acad. Sci. USA 89:1865-1869), or phage (Scott and Smith (1990) Science 249:386-390; Devlin (1990) Science 249:404-406; Cwirla et al. (1990) Proc. Natl. Acad. Sci. USA 87:6378-6382; and Felici (1991) J. Mol. Biol. 222:301-310).

In some embodiments, an assay to screen for Perforin-2 activity modulating compounds is a cell-free assay comprising contacting a polypeptide of a component of the Perforin-2 activation pathway or biologically active fragment or variant thereof with a test compound and determining the ability of the test compound to bind to a polypeptide of a component of the Perforin-2 activation pathway or the biologically active variant or fragment thereof. Binding of the test compound to a polypeptide of a component of the Perforin-2 activation pathway can be determined either directly or indirectly. In a further embodiment, the test or candidate compound specifically binds to or selectively binds to a polypeptide of a component of the Perforin-2 activation pathway.

In other embodiments, an assay comprises contacting a biological sample comprising a polypeptide of a component of the Perforin-2 activation pathway with a candidate compound and determining the ability of the candidate compound to modulate the activity of a polypeptide of a component of the Perforin-2 activation pathway. The term “biological sample” is intended to include tissues, cells, and biological fluids isolated from a subject, as well as tissues, cells, and fluids present within a subject. In some embodiments the biological sample is from lymph nodes, spleen, bone marrow, blood, or primary tumor. Determining the ability of the candidate compound to modulate the activity of a polypeptide of a component of the Perforin-2 activation pathway can be accomplished, for example, by determining the ability of the polypeptide of a component of the Perforin-2 activation pathway to activate Perforin-2, as described above, for determining Perforin-2 activity.

Further provided are novel agents identified by the above-described screening assays and uses thereof for treatments as described herein.

### IV. Sequence Identity

Active variants and fragments of the various components of the Perforin-2 activation pathway provided herein (i.e. components of the ubiquitination pathway, Perforin-2, or any Perforin-2-associated molecules thereof) can be used in the methods provided herein. Such active variants can comprise at least 65%, 70%, 75%, 80%, 85%, 90%, 91%, 92%, 93%, 94%, 95%, 96%, 97%, 98%, 99% or more sequence identity to any of the various target molecules provided herein, wherein the active variants retain biological activity and hence modulate Perforin-2 activity. A fragment of a polynucleotide that encodes a biologically active portion of a polypeptide of any of the various components of the Perforin-2 activation pathway will encode at least 15, 25, 30, 50, 100, 150, 200, 250, 300, 350, 400, 450 contiguous amino acids, or up to the total number of amino acids present in a full-length polypeptide.

As used herein, “sequence identity” or “identity” in the context of two polynucleotides or polypeptide sequences makes reference to the residues in the two sequences that are the same when aligned for maximum correspondence over a specified comparison window. When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. When sequences differ in conservative substitutions, the percent sequence identity may be adjusted upwards to correct for the conservative nature of the substitution. Sequences that differ by such conservative substitutions are said to have “sequence similarity” or “similarity”. Means for making this adjustment are well known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., as implemented in the program PC/GENE (Intelligenetics, Mountain View, Calif.).

As used herein, “percentage of sequence identity” means the value determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity.

Unless otherwise stated, sequence identity/similarity values provided herein refer to the value obtained using GAP Version 10 using the following parameters: % identity and % similarity for a nucleotide sequence using GAP Weight of 50 and Length Weight of 3, and the nwsgapdna.cmp scoring matrix; % identity and % similarity for an amino acid sequence using GAP Weight of 8 and Length Weight of 2, and the BLOSUM62 scoring matrix; or any equivalent program thereof. By “equivalent program” is intended any sequence comparison program that, for any two sequences in question, generates an alignment having identical nucleotide or amino acid residue matches and an identical percent sequence identity when compared to the corresponding alignment generated by GAP Version 10.

Non-limiting examples of the methods and compositions provided herein are as follows:

1. A method of treating a subject having inflammation of the gut comprising administering to said subject in need thereof a therapeutically effective amount of a compound that inhibits Perforin-2 activity.
2. The method of embodiment 1, wherein the subject has colitis.
3. The method of embodiment 1, wherein the subject has Crohn's disease.
4. The method of embodiment 1, wherein the subject has inflammatory bowel disease.
5. The method of any one of embodiments 1-4, wherein the compound comprises: a small molecule, a polypeptide, an oligonucleotide, a polynucleotide or combinations thereof.
6. The method of any one of embodiments 1-5, wherein the compound that inhibits Perforin-2 activity comprises an inhibitor of at least one component of the ubiquitination pathway.
7. The method of embodiment 6, wherein the compound that inhibits Perforin-2 activity comprises an E1 ubiquitin-activating enzyme inhibitor, an E2 ubiquitin-conjugating enzyme inhibitor, or an E3 ubiquitin ligase inhibitor.
8. The method of embodiment 7, wherein the compound that inhibits Perforin-2 activity comprises PYR-41, BAY 11-7082, Nutlin-3, JNJ 26854165, Thalidomide, TAME, NSC-207895, or an active derivative thereof.
9. The method of embodiment 6, wherein the compound that inhibits Perforin-2 activity comprises a Cullin Ring Ubiquitin Ligase (CRL) inhibitor.
10. The method of embodiment 5, wherein the compound that inhibits Perforin-2 activity comprises an inhibitor of the neddylation pathway.
11. The method of embodiment 10, wherein the compound that inhibits Perforin-2 activity comprises a NEDD8-activating enzyme (NAE) inhibitor.
12. The method of embodiment 11, wherein the NAE inhibitor comprises MLN-4924 or an active derivative thereof.
13. The method of any one of embodiments 1-5, wherein the compound that inhibits Perforin-2 activity comprises a deamidase.
14. The method of embodiment 13, wherein the deamidase comprises Cif.
15. The method of any one of embodiments 1-4, wherein the compound that inhibits Perforin-2 activity comprises a proteasome inhibitor.
16. The method of embodiment 15, wherein the proteasome inhibitor comprises Bortezomib, Salinosporamide A, Carfilzomib, MLN9708, Delanzomib, or an active derivative thereof.
17. A method of increasing Perforin-2 activity comprising: administering to a subject in need thereof, a therapeutically effective amount of at least one compound which increases the ubiquitination of Perforin-2; and, thereby increasing the activity of Perforin-2.
18. The method of embodiment 17, wherein the at least one compound increases the activity and/or expression of at least one component of the ubiquitination pathway.
19. The method of embodiment 18, wherein the at least one component of the ubiquitination pathway comprises an E1 ubiquitin-activating enzyme, an E2 ubiquitin-conjugating enzyme or an E3 ubiquitin ligase.
20. The method of embodiment 17, wherein the at least one compound comprises an isopeptidase inhibitor.
21. The method of embodiment 20, wherein said isopeptidase inhibitor comprises Ubiquitin Isopeptidase Inhibitor II (F6) (3,5-bis((4-Methylphenyl)methylene)-1,1-dioxide, piperidin-4-one), Ubiquitin Isopeptidase Inhibitor I (G5) (3,5-bis((4-Nitrophenyl)methylene)-1,1-dioxide, tetrahydro-4H-thiopyran-4-one) or an active derivative thereof.
22. The method of embodiment 17, wherein the at least one compound comprises a deubiquitinase inhibitor.
23. The method of embodiment 22, wherein the deubiquitinase inhibitor comprises PR-619, IU1, NSC 632839, P5091, p22077, WP1130, LDN-57444, TCID, b-AP15 or an active derivative thereof.
24. The method of embodiment 17, wherein the at least one compound comprises a deneddylation inhibitor.
25. The method of embodiment 24, wherein the deneddylation inhibitor comprises PR-619, Ubiquitin Isopeptidase Inhibitor II (F6) (3,5-bis((4-Methylphenyl)methylene)-1,1-dioxide, piperidin-4-one), Ubiquitin Isopeptidase Inhibitor I (G5) (3,5-bis((4-Nitrophenyl)methylene)-1,1-dioxide, tetrahydro-4H-thiopyran-4-one) or an active derivative thereof.
26. The method of any one of embodiments 17-25, wherein the at least one compound inhibits replication, inhibits growth, or induces death of an infectious disease organism.
27. The method of embodiment 26, wherein the infectious disease organism is an intracellular bacterium.
28. A method of treating a subject suffering from an infectious disease organism comprising, administering to the subject a therapeutically effective amount of at least one compound that increases the activity of Perforin-2, wherein said compound increases the ubiquitination of Perforin-2.
29. The method of embodiment 28, wherein the at least one compound increases the activity or expression of at least one component of the ubiquitination pathway.

As used herein, the singular terms “a,” “an,” and “the” include plural referents unless context clearly indicates otherwise. Similarly, the word “or” is intended to include “and” unless the context clearly indicates otherwise. It is further to be understood that all base sizes or amino acid sizes, and all molecular weight or molecular mass values, given for nucleic acids or polypeptides are approximate, and are provided for description.

The subject matter of the present disclosure is further illustrated by the following non-limiting examples.

### EXPERIMENTAL

### Example 1

### PERFORIN-2: A Novel and Critical Effector to Eliminate Intracellular Bacteria

Perforin-2 (P-2) is an innate effector molecule of unique importance to destroy invading bacteria by physical attack. Upon polymerization P-2 forms clusters of large holes and pores in the cell wall/envelop of bacteria that impair the barrier function and permit entry of reactive oxygen and nitrogen species and hydrolases to complete bacterial destruction. In the absence of P-2, ROS, NO and lysozyme have minimal bactericidal activity.

Perforin-2 is expressed or induced ubiquitously in all phagocytic and non-phagocytic human and mouse cells and cell lines tested and required to eliminate intracellular bacteria.

Perforin-2 is highly conserved through evolution from sponges (Porifera) to humans (Homo).

The deficiency of Perforin-2 in mice renders them defenseless to orogastric infection with Salmonella typhimurium or epicutaneous infection with Staphylococcus aureus or vaginal Chlamydia infections. The P2−/− mice die from infections that are cleared by P-2+/+ litter mates.

All non-phagocytic and phagocytic cells in mice and humans express P-2 upon induction.

P-2 knock-down or deficiency renders cells including macrophages and PMN defenseless and unable to kill intracellular bacteria resulting in intracellular bacterial replication that kills the cells.

It is important to determine that human P-2 is of equal importance in killing bacteria as has been established in mice in vivo and in vitro.

Human P-2 in vitro, in cell lines has the same critical importance as in mouse cell lines.

The main ports of entry for bacterial infections are the mucosal surfaces and the skin. We will study the role of P-2 in keratinocytes and in intestinal epithelial cells in normal cells, in patients with wound healing defects and in patients with inflammatory bowel disease.

Bacteria have evolved ways to suppress, block or evade P-2. For instance Chlamydia is able to suppress P-2 mRNA induction in mucosal epithelial cells (HeLa) in vitro and in vaginal cells in mice in vivo. Cif plasmid in enteropathogenic E. coli can block P-2 killing by blocking P-2-polymenrization. To stop bacteria from blocking P-2 it is necessary to understand the pathway by which P-2 is activated in human cells and to develop drugs that counteract the bacterial factors.

Perforin-2 has not been studied in humans although its expression at the mRNA level has been known as macrophage expressed gene 1.

The discovery of the unique functions of P-2 in anti-bacterial defense creates a new paradigm in innate immunity. New drugs and methods will be developed based on the function of P-2 to defeat difficult bacterial infections.

It is likely that bacteria that have taken up residence in human cells, even if only temporarily, must have evaded or blocked P-2. This includes antibiotic resistant bacterial infections—by virtue of residing in human cells the bacteria must have been able to neutralize the ability of P-2 to kill them. Counteracting P-2-resistance factors of the bacteria causing infection is expected to allow P-2 to kill the disease causing bacterium.

Bacterial factors resisting P-2 will be distinct from factors providing antibiotic resistance due to the vastly different nature of anti-bacterial attack by antibiotics—namely chemical attack—and P-2, which attacks by physical attack and generates large defects in the bacterial envelop. The defects in the envelop allows secondary mediators, lysozyme, ROS and NO to penetrate and cause bacterial lysis.

The Role of Perforin-2 (P-2) in Bacterial Infections in Skin and Mucosa

The skin and mucosa are the major entry sites for bacterial infections. Our new data on structure and function of P-2 indicate that P-2 is the earliest innate anti-bacterial effector that is required to kill and eliminate intracellular bacteria in phagocytic and non-phagocytic cells. Moreover, P-2 is also essential to initiate the inflammatory response that appears to be essential to clear pathogens. P-2 deficiency is associated with lethal outcome upon infection of skin or mucosa with pathogenic bacteria. On the other hand inappropriate P-2 activation and bacterial killing can cause inflammation and morbidity that may be responsible for some auto-aggressive syndromes.

We will study for the first time this novel effector pathway with particular emphasis on the skin and the intestinal mucosa and associated diseases. In addition the new information will be used for forays into novel drug development to defeat bacterial infections.

Introduction:

Our group has studied a novel anti-bacterial effector protein in mice and humans, designated Perforin-2 (P-2), owing to its ‘perforating’ function that generates clusters of large holes (100 Å diameter) or “pores” in bacterial envelops. The perforating function is essential to kill intracellular bacteria including Mycobacteria, Gram-positive and Gram negative bacteria also including Listeria monocytogenes, Shigella Flexneri and obligate intracellular Chlamydia trachomatis (data not shown). The traditional bactericidal effectors ROS, NO and hydrolytic enzymes including lysozyme strongly enhance the bactericidal activity of P-2 but are unable to block intracellular replication of bacteria in the absence of P-2.

In order to replicate, bacteria frequently invade tissue epithelial cells and other non-phagocytic cells. Importantly, we found that all cells can express P-2 and that P-2-knock-down abrogates the cells' ability to block intracellular bacterial replication. Perforin-2 thus appears to be a dominant anti-bacterial effector in mice and humans in all non-phagocytic and phagocytic cells that is critical for health.

The skin and mucosal surfaces are the sites exposed to and frequently invaded by pathogenic bacteria. Studies in P-2-deficient mice generated by our group confirmed the critical role of P-2 in antibacterial defense in vivo in mucosal and in skin infection models. P-2 deficient mice died of infections that are cleared by P-2 sufficient littermates.

Evolutionary studies indicate that Perforin-2 is an ancient anti-bacterial mechanism, known as mpeg1, that is highly conserved from sponges (Porifera) to mammals including humans. Our data in mice and in humans indicate that P-2 constitutes a crucial anti-bacterial effector mechanism that requires detailed study in human disease. Understanding the molecular mechanisms by which bacterial pathogens interfere with or evade P-2 will point the way to develop novel treatment to combat antibiotic resistant bacterial infections.

1. Structure of Perforin-2 and Mechanism of Activation

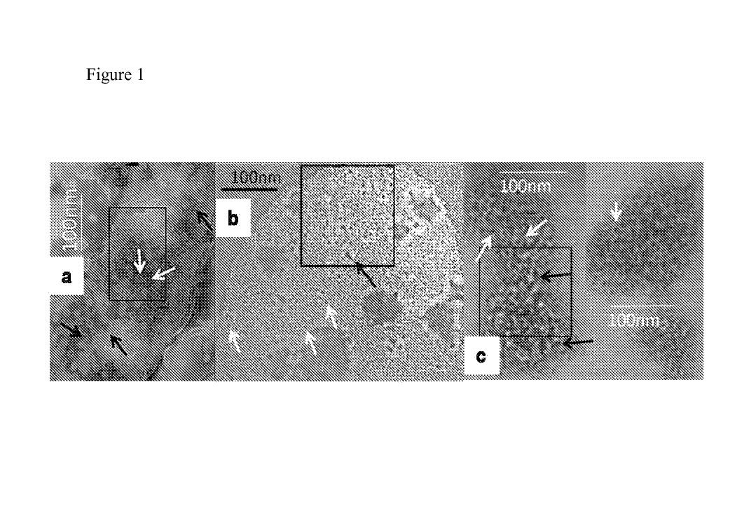

Perforin-2 is an integral transmembrane protein stored in membrane vesicles in the cytosol. Perforin-2 contains a Membrane Attack Complex Perforin domain (MACPF) which is found in the pore-forming proteins of complement including poly-C9 and in Perforin-1. The MACPF domains of C9 and Perforin-1 are responsible for pore-formation by refolding two a-helical sequences into amphiphilic β-sheets that polymerize while inserting into bacterial cell walls and forming clustered amphiphilic β-barrels that disrupt the structure of the bacterial envelop. We have imaged by electron microscopy human poly-P-2 clusters in eukaryotic bilayer membranes and mouse poly-P-2 in bacterial cell walls (MRSA and Mycobacterium smegmatis) and found that the inner diameter of poly-P-2 pore is 90-100 Å (FIG. 1) which is similar in size to the MAC-poly C9 complex of complement but smaller than poly-P-1 (160 Å).

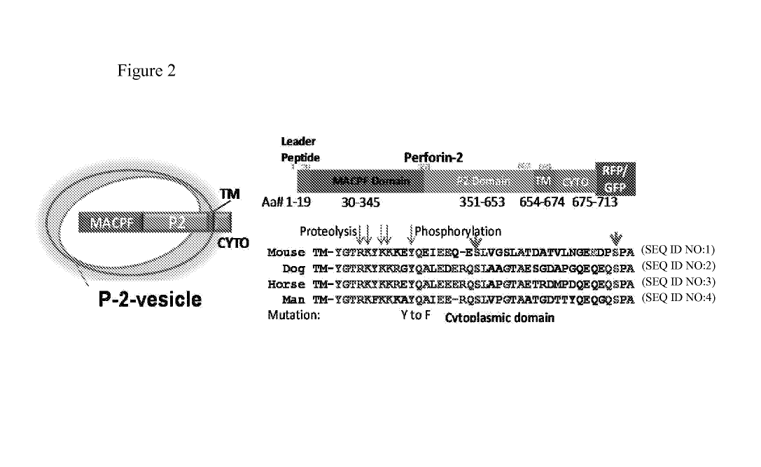

Activation of P-2: As mentioned above P-2 is a transmembrane protein; the N-terminal MACPF domain of P-2 is located in the lumen of membrane vesicles, the C-terminus terminates in a short, 36 amino acid cytoplasmic domain (FIG. 2).

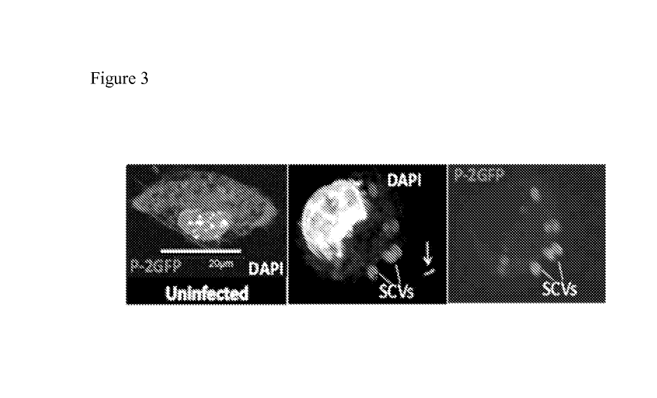

After infection of cells bacteria are contained in an endosomal or phagosomal membrane vesicle, known as bacterium containing vacuole (BCV). The location of the MACPF at the N-terminus of P-2 and its orientation pointing into the lumen of cytosolic membrane vesicles is ideal for killing bacteria inside vacuoles by polymerization and insertion of the MACPF domain into the bacterial envelope. This requires the translocation of P-2-bearing vesicles that are stored in the cytosol to and fusion with the BCV. This is indeed the case as is shown in FIG. 3, where GFP marked P-2 (P-2-GFP) is found on the Salmonella containing vacuole (SCV) within 5 min of infection. Moreover, translocation of P-2-GFP to the SCV is associated with DNA release from Salmonella as detected by DAPI staining (shown in white) suggesting killing by P-2 (FIG. 3).

The conserved cytoplasmic domain of P-2 (FIG. 2) suggests that it may interact with proteins that control P-2-vesicle translocation and P-2 polymerization. Using the P-2 two hybrid screen, P-2-coimmunoprecipitation, co-translocation with P-2-GFP to the SCV, knock down by siRNA to inhibit bactericidal activity and use of chemical inhibitors we have identified some of the proteins that are essential for P-2 activity in killing intracellular bacteria (Table 1).

TABLE 1
Proteins interacting with the cytoplasmic domain of P-2. Several
assays were used for determine and validate P-2-interaction as
indicated, but not all assays for all interacting proteins.

| | Yeast | P-2- | | | | |
|---|---|---|---|---|---|---|
| | two | coimmune | P-2- | | | NEDDS |
| | hy- | precip- | cotrans- | | Chemical | blocking |
| Protein | brid | itation | location | siRNA | inhibitor | plasmid |
| RASA2 | + | + | + | + | | |
| Vps34 | | + | | | + | |
| Ubc12 | + | + | | + | | |
| NEDDS | | | | | | + |
| Cullin1 | | + | | | | |
| βTrCP1,2 | | + | | | | |

2. Molecular Mechanisms of P-2 Activation:

a. Phosphorylation: Based on the phylogenetic conservation of Y and S in P-2-cyto shown in FIG. 2, it is likely that phosphorylation of serine and tyrosine is one of the first activation signals triggered by bacterial endocytosis. Kinase candidates are TEC, NEK9 and Mapk12

b. Translocation: Next translocation of P-2-vesicles (see FIG. 3) to the bacterium containing vacuole is likely to require the PI3-kinase vps34 and RASA2/GAP1M which interact with the cytoplasmic domain of P2.

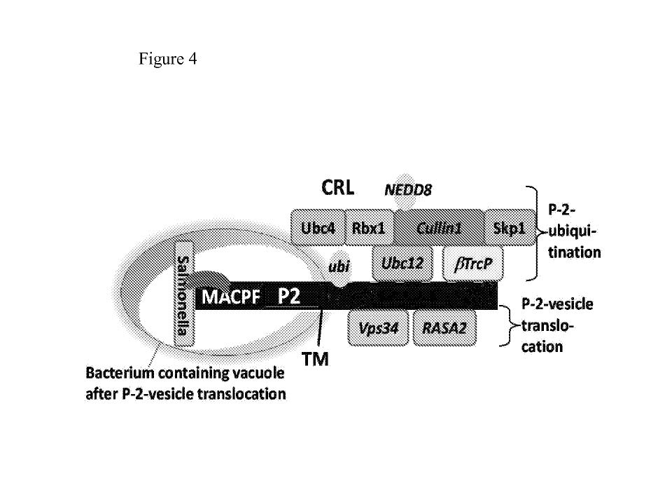

c. P-2-ubiquitination, polymerization and killing: Following P-2-vesicle translocation and fusion with the bacterium containing vacuole, P-2 needs to be activated to polymerize and attack the bacterial envelope inside the vacuole. We suggest that P-2 is ubiquitylated at the lysine cluster (FIG. 2) which attracts proteasomes to degrade the cytoplasmic domain and allows P-2 to align in such a way that it can polymerize and attack the bacterium by insertion of MACPF-sequences that form the amphiphilic β-barrel disrupting the integrity of the envelope (see FIG. 1). P-2 ubiquitylation is carried out by a Cullin-Ring-ubiquitin-Ligase (CRL) composed of the substrate recognition unit βTrCP bound to the adapter Skp1-Cullin1-Rbx1-Ubc(4) (CRL1^{βTrCP}) (P-2 signaling complex, FIG. 4). βTrCP and cullin1 coimmunoprecipitate with P-2 (Table 1).

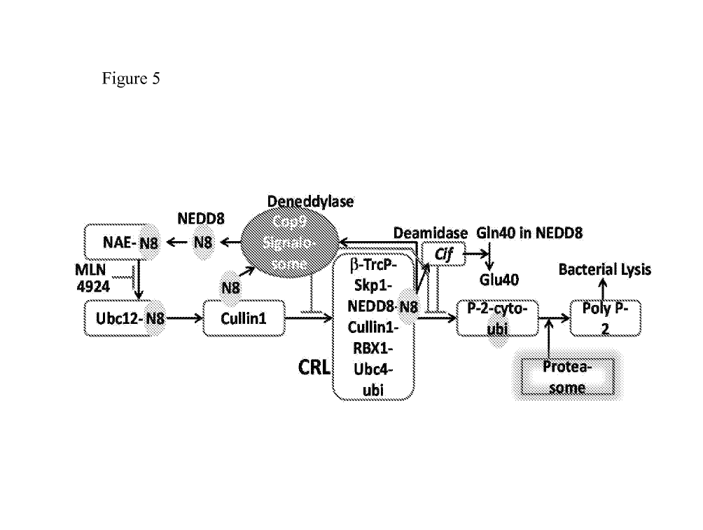

All CRLs require activation by ligation of NEDD8 to cullins. NEDD8 is activated by the E1-ligase, NEDD8 activating enzyme-1 (NAE1), transferring NEDD8 to the E2 ligase ubc12 which in turn neddylates cullin1 that via RBX1 activates the ubiquitin ligase (ubc4) to ubiquitylate P-2. We have shown that ubc12 interacts with P-2 by yeast two hybrid analysis and coimmunoprecipitates with P-2. NEDD8 is inactivated by the Cif-plasmid deamidating Gln40 of NEDD8 to Glu40. NEDD inactivation protects bacteria from being killed by P-2. FIG. 5 shows the pathway of neddylation and deneddylation that controls CRL activity and P-2 activation.

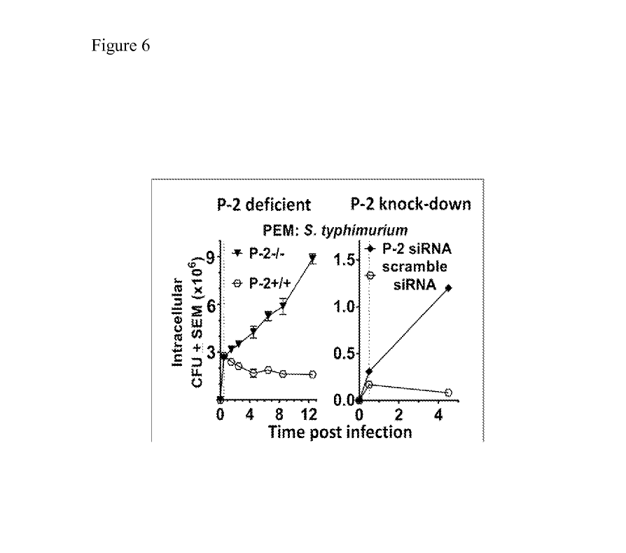

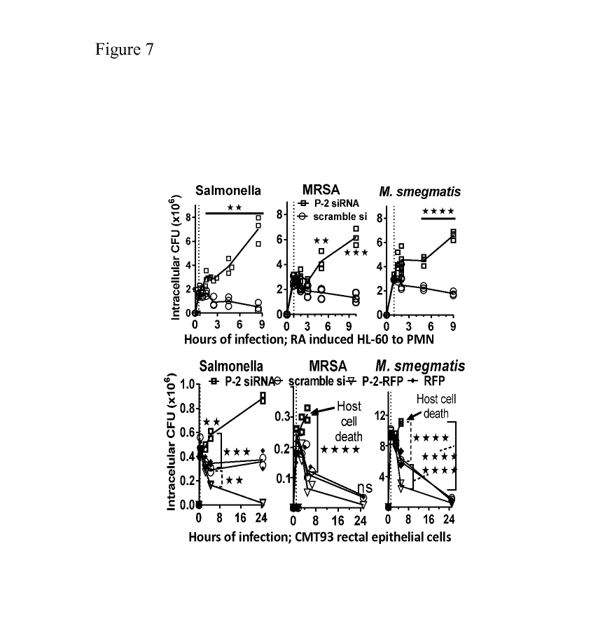

3. P-2 depletion and the role of ROS, NO and lysozyme in bactericidal activity. Genetically P-2 deficient or siRNA P-2 depleted peritoneal macrophages are unable to kill S. typhimurium and unable to prevent their intracellular replication (FIG. 6). In addition they are also unable to control MRSA and M. smegmatis (not shown). P-2 siRNA knock down was used in other cells with identical results: when P-2 is knocked down the cells are unable to control intracellular infection by Salmonella, MRSA or M. smegmatis as shown in FIG. 7 for PMN, generated by retinoic acid induction in HL60 or in CMT93 rectal epithelial cells (carcinoma). P-2 overexpression by P-2-GFP transfection in addition to endogenous P-2 increases anti-bacterial activity. The data suggested that P-2 is absolutely required to control intracellular bacterial infection and that ROS, NO and lysozyme is unable to do so without P-2.

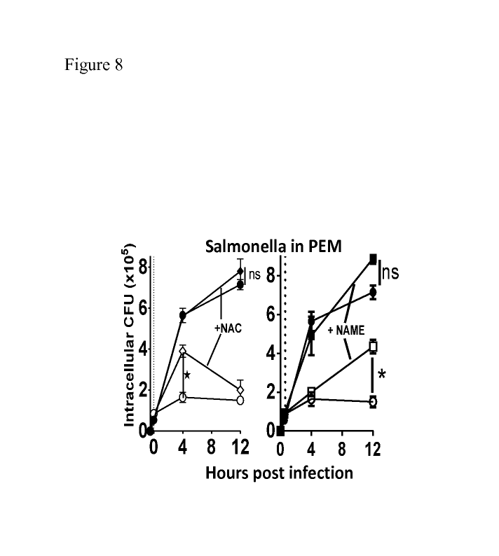

The analysis of ROS, NO and P-2 in their ability to kill intracellular Salmonella in IFN-γ activated, thioglycolate elicited, peritoneal macrophages (FIG. 8) indicated that ROS and NO together in the absence of P-2 are unable to significantly delay intracellular bacterial replication. In the presence of P-2, ROS contributes to the bactericidal activity during the first 4 hours of infection. After 4 h the effect of NO contributing to P-2 bactericidal activity becomes evident (FIG. 8). The data clearly indicate that ROS and NO require the presence of P-2 mediated damage to the bacterial envelop for their full bactericidal activity. We interpret these data to indicate that the penetration of ROS and NO to sensitive sites becomes possible after physical damage to the integrity of the bacterial envelop by P-2 polymerization and formation of clustered holes and pores (see FIG. 1). We have found that lysozyme, too, is bactericidal only after prior damage of the envelope by P-2 in murine embryonic fibroblasts (MEF). The mechanism is analogous to Peforin-1 attacking virus infected or cancer cells and providing entry for granzymes to mediate their cytotoxic activity.

Our data indicate that damage to the bacterial envelop inflicted by P-2-polymerization is necessary to mediate the bactericidal effects of other antibacterial effectors. In the absence of Perforin-2 intracellular bacteria of three major subgroups (Gram-positive, -negative and acid fast) are no longer killed and replicate undeterred despite the presence of other bactericidal mediators. These data alter the current paradigm of anti-bacterial effector mechanisms.

We have also established that human cells express P-2 and that it is required to prevent intracellular replication of bacteria (FIG. 7 upper panel). However the molecular details of the activation of human P-2 are not known.

4. Expression and Induction of Perforin-2

P-2 is expressed ubiquitously in all human and mouse cells tested from all lineages of endoderm, ectoderm, mesoderm and neuroectoderm (Tables 2 and 3). P-2 expressing cells include but are not restricted to myoblasts, neuroblasts, astrocytes, melanocytes, pancreatic glandular cells, uroepthelial cells, intestinal columnar epithelial cells, cervical epithelial cells, keratinocytes, endothelial cells, kidney epithelial cells, fibroblasts, in addition to phagocytic cells including polymorphonuclear neutrophilic granulocytes (PMN), macrophages, dendritic cells, microglia and lymphocytes. Expression of P-2 by non-phagocytic cells is induced rapidly, within 6-8 hours, by IFN α, β or γ, or by intracellular bacterial infection. In phagocytic cells including PMN and in keratinocytes P-2 is expressed constitutively and further up-regulated by IFN and LPS.

TABLE 2
Expression of Perforin-2 in Human Cells

| Cell type | Perforin-2 mRNA | Perforin-2 |
|---|---|---|
| H.s.—Homo sapiens | status? | killing? |
| Monocyte Derived Macrophage (H.s.) | Constitutive | Yes |
| Polymorphonuclear granulocyte (H.s.) | Constitutive | N.D. |
| HL-60 promyelocyte → PMN (H.s) | Constitutive | Yes |
| Primary keratinocytes (H.s) | Constitutive | N.D. |
| Umbilical endothelial cells (H.s.) | Inducible | Yes |
| HeLa cervical carcinoma (H.s.) | Inducible | Yes |
| UM-UC-3 bladder Cancer (H.s) | Inducible | Yes |
| UM-UC-9 bladder Cancer (H.s) | Inducible | Yes |
| HEK-293 embryonal kidney (H.s.) | Inducible | Yes |
| MIA-PaCa-2 pancreatic cancer (H.s) | Inducible | Yes |

TABLE 3
Expression of Perforin-2 in Murine Cells.

| Cell type | Perforin-2 mRNA | Perforin-2 |
|---|---|---|
| M.m.—Mus musculus | status? | killing? |
| Peritoneal macrophages | Constitutive | Yes |
| Bone marrow derived DC | Constitutive | Yes |
| BV-2 Microglia | Constitutive | Yes |
| CATH.a neuroblastoma | Inducible | Yes |
| Neuro-2A neuroblastoma | Inducible | Yes |
| Primary CNS fibroblast | Inducible | Yes |
| Primary astrocytes | Inducible | Yes |
| Murine embryonic fibroblast | Inducible | Yes |
| NIH/3T3 fibroblast | Inducible | Yes |
| C2C12 myoblast | Inducible | Yes |
| CMT-93 rectal carcinoma | Inducible | Yes |
| CT26 colon carcinoma | Inducible | Yes |
| B16-F10 melanoma | Inducible | Yes |
| MOVCAR 5009 Ovarian Carcinoma | Inducible | Yes |
| MOVCAR 5447 Ovarian Caricinoma | Inducible | Yes |

Human P-2 is encoded on chromosome 1 by mpeg1 (macrophage expressed gene 1). The entire ORF and part of the 5′ and 3′ untranslated sequence is contained a single exon of ˜4.5 kb, a second short exon encoding the 5′ start. The chromosomal locus is wide open in more than 125 cell lines as analyzed by DNAse hypersensitivity assays in the ENCODE project. About 4 kb upstream of transcription start is al DNAse I hypersensitivity cluster which is associated with 29 transcription factors identified by chromatin immunoprecipitation (CHIP) assays. The strongest signals in the Chip assay come from Pu.1, BATF, NFκB, Oct-2, POU2F2, PAX5, RXRA, BCL11, IRF4, TCF12, BCL3 and p300. These data suggest that the locus is open and ready to be transcribed rapidly as is indeed observed in all cells analyzed.

5. In Vivo Analysis of P-2 by Bacterial Challenge of P-2 Deficient Mice.

We have generated genetic P-2 deficiency in mice by homologous gene replacement. P-2 deficient cells, for instance P-2 deficient, elicited peritoneal macrophages or embryonic fibroblasts (MEF), are unable to prevent intracellular bacterial replication (see FIG. 6). We have challenged P-2−/− in three disease models.

5.1 Staphylococcus aureus (MRSA): P-2−/− mice develop and thrive normally. The composition of their cellular immune repertoire is normal including all myeloid and lymphoid cell populations in blood and spleen (data not shown) indicating a normal adaptive and innate immune system but lacking the P-2 effector protein.

In the epicutaneous mouse skin infection model the barrier of the shaved skin is disrupted by tape stripping removing most of the protective corneal layer. One cm² of skin is then exposed to MRSA and bandaged for the next 24 h causing local infection and inflammation characterized by IL-6, TNF-α and IFN-γ production and production of the mouse β-defensins mBD3 and mBD4.

P-2−/− mice were challenged epicutaneously with methicillin resistant Staphylococcus aureus (MRSA), clinical isolate CLP148. P-2−/− mice rapidly lose weight requiring euthanasia (IACUC requirement) suggesting that they would die. In contrast P-2+/+ and P-2+/− mice do not lose weight and appear healthy except for the signs of local skin infection. Analyzing colony forming units (cfu), P-2−/− mice have high counts in blood, kidney, spleen and skin in contrast to P-2+/+ mice that have high counts only in the skin at the infection site. P-2+/− mice have intermediate cfu counts. The data suggest that P-2 expressed constitutively by keratinocytes in the epidermis may be important for protection from infection and invasion by Staphylococci and probably other bacteria.

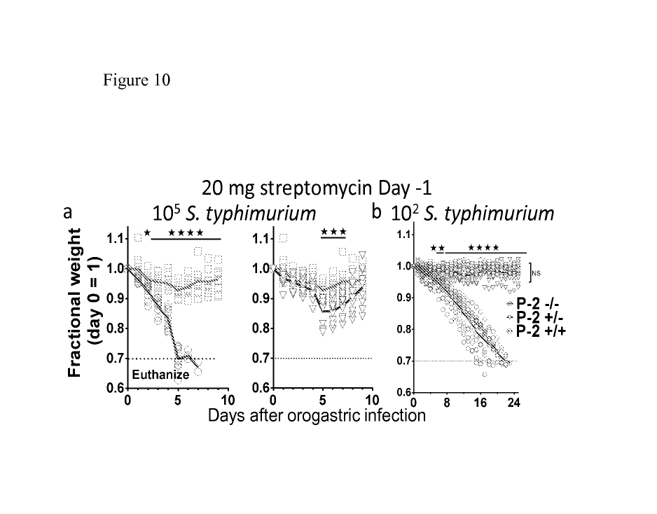

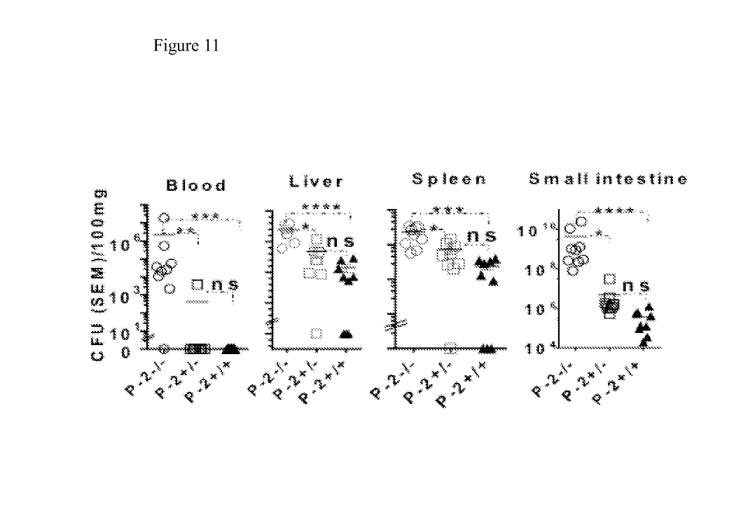

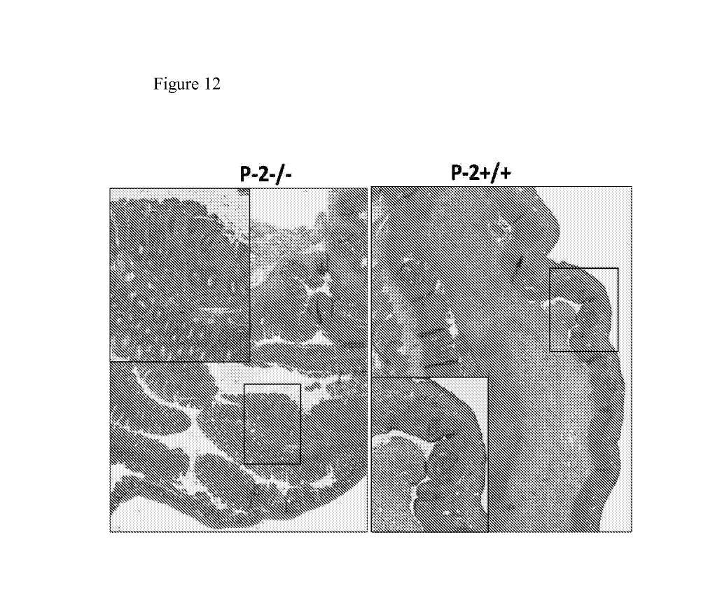

5.1 Salmonella typhimurium: Salmonella typhimurium is a human pathogen. We challenged P-2−/− mice and litter mates with S. typhimurium (RL144, gift of Dr. Galan, Yale University) by the orogastric route according to established protocols. P-2−/− mice die after orogastric challenge with 10⁵ or 10² S. typhimurium that are cleared by P-2+/+ or P-2+/− litter mates (FIG. 10). P-2−/− but not P-2+/+ mice have high level bacteremia indicating bacterial dissemination (FIG. 11). Strikingly, however, by histopathology P-2−/− show barely any signs of inflammation in the cecum/colon while P-2+/+ mice exhibit massive inflammation associated with PMN and mononuclear infiltration, necrosis, loss of goblet cells, submucosal edema and hyper-proliferation (FIG. 12). The data indicate that P-2 mediated killing of Salmonella releases large amounts of pathogen associated patterns (PAMPS) that cause the inflammation that contributes to clearance.

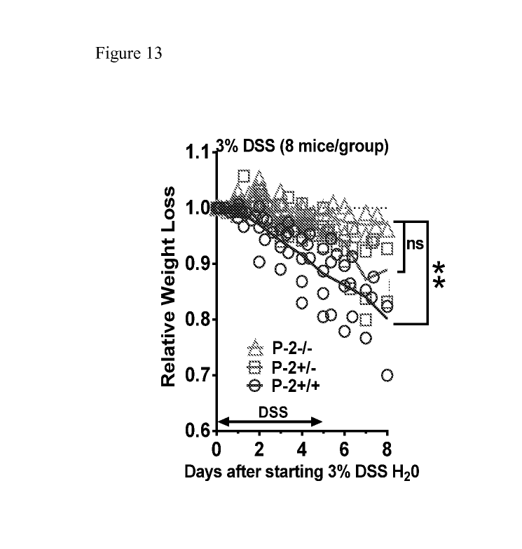

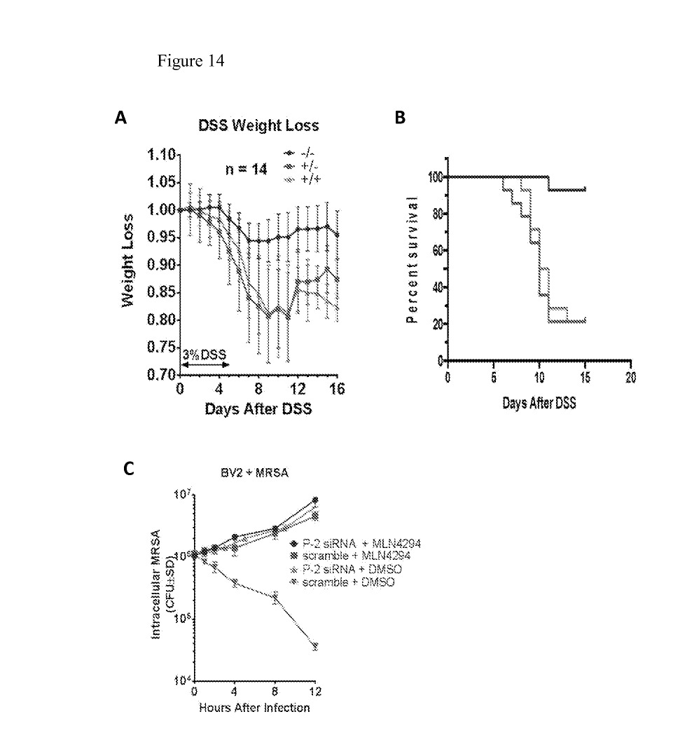

Dextran sodium sulfate (DSS) colitis: Challenging P-2+/+ and P-2−/− in the inflammatory bowel disease model with 3% dextran sodium sulfate (DSS), we found that P-2−/− mice do not lose weight and do not acquire diarrhea while P-2+/+ littermates have massive diarrhea, bloody stools and severe weight loss (FIGS. 13 and 14). However the blood remains sterile in both, P-2+/+ and P-2−/− mice indicating that the commensal bacteria cause inflammation but are not invasive. In histopathology, P-2+/+ mice show massive inflammation and necrosis as expected. P-2−/− have no inflammation (data not shown). The data suggest that DSS damages the mucus layer and the epithelial cells resulting in intimate contact of commensal bacteria with cell membranes. Cell contact causes endocytosis of bacteria, P-2-activation and bacterial killing with release of PAMPs from commensal bacteria that initiate the inflammatory response. In the absence of P-2, commensals are not killed, PAMPs are not released and no inflammation ensues. The data suggest that inflammatory bowel disease may be initiated by P-2 when the normal mucus layer or epithelial cells in cecum and colon are damaged.

### Example 2

### A. Increasing Perforin-2 Expression

Human P-2 is encoded on chromosome 1 by mpeg1 (macrophage expressed gene 1). The entire ORF and part of the 5′ and 3′ untranslated sequence is contained a single exon of ˜4.5 kb, a second short exon encoding the 5′ start. The chromosomal locus is wide open in more than 125 cell lines as analyzed by DNAse hypersensitivity assays in the ENCODE project. About 4 kb upstream of transcription start is a DNAse I hypersensitivity cluster which is associated with 29 transcription factors identified by chromatin immunoprecipitation (CHIP) assays. The strongest signals in the Chip assay come from Pu.1, BATF, NFκB, Oct-2, POU2F2, PAX5, RXRA, BCL11, IRF4, TCF12, BCL3 and p300. These data suggest that the locus is open and ready to be transcribed rapidly as is indeed observed in all cells analyzed.

Any drug that increases P-2 transcription will increase P-2 expression and enhance bacterial clearance. Since the P-2 locus is wide open it is straight forward to determine P-2 transcription or set up P-2 reporter assays and screen drugs for activity.

### B. Increasing P-2 Activity

P-2 activation requires translocation to the bacterium containing vacuole and activation for P-2-polymerization and anti-bacterial attack by a cullin-ring-ubiquitin-ligase (CRL) using the P-2 recognition component βTrCP1/2.

Translocation is mediated by RASA2 and vps34. Activation for polymerization and killing requires several proteins including ubc12, NEDD8, cullin-1, Rbx1, Skp1 and βTrCP1/2 to form the complex of the Cullin-ring-ubiquitin-ligase (CRL) required for P-2 ubiquitylation and proteasome mediated degradation of the P-2 cytoplasmic domain.

Any drug that enhances expression levels of the CRL components or enhances their complex formation or increases CRL half-life is expected to increase P-2 activation.

CRLs are deneddylated by the Cop-9 signalosome; Csn5 is the active isopeptidase component of Cop-9 responsible for deneddylation Inhibition of Csn5 with isopeptidase inhibitors is expected to increase the half-life of the CRL required for P-2 ubiquitylation and increase anti-bacterial activity.

### C. Inhibiting P-2 Activity

Our data in the Dextran-sodium sulfate (DSS)-colitis model in P-2−/− mice show that P-2 is required for induction of inflammation in the colon upon DSS administration. P-2 mediated killing of bacteria can be inhibited with inhibitors of NEDD8 ligation to cullin1. We have tested inhibitors of the NEDD8 activating enzyme NAE1 with MLN 4924, and found that it blocks P-2 mediated bacterial killing in vitro (FIG. 14c). This indicates that P-2 inhibitors will be useful for the treatment of Crohn's colitis, Ulcerative Colitis and inflammatory bowel disease. Moreover, P-2 inhibition may be beneficial for disorders that are initiated by deregulated or excessive activity of P-2.

### Example 3

We have identified a novel effector pathway, named Perforin-2 that is expressed constitutively in all phagocytic and inducibly in all non-phagocytic cells tested to date. Perforin-2 is essential for the killing of pathogenic, intracellular bacteria (3). Genetically Perforin-2 deficient cells including Perforin-2−/− mouse embryonic fibroblasts, macrophages and polymorphonuclear neutrophils (PMN) are unable to clear intracellular bacterial infection with Gram-positive (MRSA), Gram-negative (Salmonella, enteropathogenic E. coli [EPEC]) bacteria, or Mycobacteria (M. smegmatis, M. tuberculosis [Mtb] and M. avium) and obligate intracellular Chlamydiae (4). Similarly, siRNA knock down of Perforin-2 blocks killing and enables intracellular replication of bacteria in macrophages, PMN and non-phagocytic cells (3). Survival of intracellular bacteria and intracellular replication requires that the bacteria silence or evade Perforin-2. Mycobacterium tuberculosis (Mtb) is an intracellular human pathogen of enormous clinical importance representing a significant scientific challenge. We have incontrovertible evidence that Perforin-2 can kill intracellular Mycobacteria including Mtb. But we have also evidence that Mycobacteria have powerful Perforin-2 resistance mechanisms. We have defined the basic steps in Perforin-2 activation for killing of intracellular bacteria and identified the steps that can potentially be blocked by bacteria to escape Perforin-2 mediated death. These steps are blockade of: (1) Perforin-2 induction and expression; (2) Perforin-2-translocation to the bacterium containing vacuole and (3) triggering for Perforin-2-polymerization, pore formation and bacterial killing. We will identify the steps of Perforin-2 expression and/or activation that are inhibited by Mtb (and by M. avium and M. smegmatis as surrogates) and to begin identifying the Mtb genes responsible for Perforin-2 inhibition. These studies will yield new scientific insights and point the way to develop effective ways to block the devastating disease of tuberculosis. Perforin-2 is an entirely novel anti-bacterial pathway that we have been studying in mice and humans. Perforin-2 is a consensus MACPF-domain containing protein (5-7) suggesting that it can kill by pore-formation via the MACPF domain (2) similar to poly-Perforin-1 of CTL and poly-C9 complement, both of which we have identified and characterized as pore-forming proteins several years ago (8, 9). We have shown by electron microscopy that Perforin-2 also is a pore forming protein and that it forms large clusters of connected pores on 6% or more of the surface area of killed intracellular MRSA and Mycobacterium smegmatis and that it significantly interferes with intracellular replication of activated macrophages. We have also shown that all phagocytic cells tested including PMN macrophages and microglia and keratinocytes express Perforin-2 constitutively. Moreover, all non-phagocytic cells tested in mice and humans (see tables 2 and 3) can be induced by IFN-α, β or γ or by microbial products to express Perforin-2. When Perforin-2 is knocked down or genetically deleted intracellular bacteria replicate rapidly and kill the invaded cells. This statement is true for phagocytes including PMN and non-phagocytic cells even after IFN treatment. This statement is also true regardless of the type of invading bacteria. We have verified this dependence on Perforin-2 for killing of Gram positive methicillin resistant Staphylococcus aureus (MRSA), Listeria monocytogenes, Gram negative Salmonella typhimurium, enteropathogenic E. coli, Yersinia pseudotuberculosis, Shigella flexneri, Mtb, M. smegmatis and M. avium, Pseudomonas aeroginosa and for obligate intracellular Chlamydia (4). The data indicate that Perforin-2 is a dominant bactericidal effector active against intracellular bacteria. Moreover, reactive oxygen and nitrogen species and hydrolases including lysozyme are synergistic with but require the membrane damaging activity of Perforin-2 for their full bactericidal force.

Experimental Approach:

Our previous data (3, 4) and preliminary data further described below indicate that killing and elimination of pathogenic, intracellular bacteria requires the function of Perforin-2. Furthermore the bactericidal functions of ROS, NO, and lysozyme depend on or are greatly enhanced by clusters of clustered pores generated by Perforin-2 on the bacterial surface. Therefore, pathogenic bacteria replicating inside cells must have found ways to block, suppress or evade Perforin-2. The evasion from Perforin-2 mediated killing simultaneously provides protection from ROS, NO and lysozyme that largely depend for their function on physical damage (perforation) of the surface of the bacterial envelop (3).

Mycobacterium tuberculosis is a major pathogen causing about 1.1 million deaths annually worldwide. Upon infection the mycobacteria are phagocytosed by macrophages but survive and replicate intracellularly and cause disease. We postulate that Mtb suppresses, evades or blocks Perforin-2; we further postulate that counteracting the mycobacterial strategy for Perforin-2 evasion will allow clearance of the bacteria. We will determine how intracellular Mycobacteria interfere with or evade Perforin-2. The primary focus is Mtb, the primary pathogen. However we will also study M. avium and M. smegmatis as surrogate (for experimental ease) and for comparison (to observe specialization of Mtb).

Experimental strategy: Perforin-2 mediated killing of intracellular bacteria includes a cascade of activation steps for targeting and translocation and ultimately killing by clustered pore formation by Perforin-2 on the bacterial envelop. To escape death bacteria have the option of blocking Perforin-2 at any step in the activation cascade. Before we can devise a counter strategy, we first have to determine which step is blocked. This will be accomplished with Mtb and compared to M. smegmatis and avium.

I. What are the Molecular Mechanisms by which Mycobacteria Interfere with Perforin-2 Expression?

Many pathogenic bacteria invade preferentially non-phagocytic cells. For instance Chlamydiae establish productive infection in epithelial cells but are unable to do so in macrophages. Salmonella, enteropathogenic E. coli (EPEC), Yersinia pseudotuberculosis attack columnar epithelial cells. Mycobacteria invade and replicate in macrophages and non-phagocytic cells. MRSA attack keratinocytes. Published data indicate that all cells can potentially be invaded by bacteria and may have mechanisms for bacterial rejection. Our data suggest that Perforin-2 may be the innate bactericidal effector molecule used by all cells to kill intracellular bacteria.

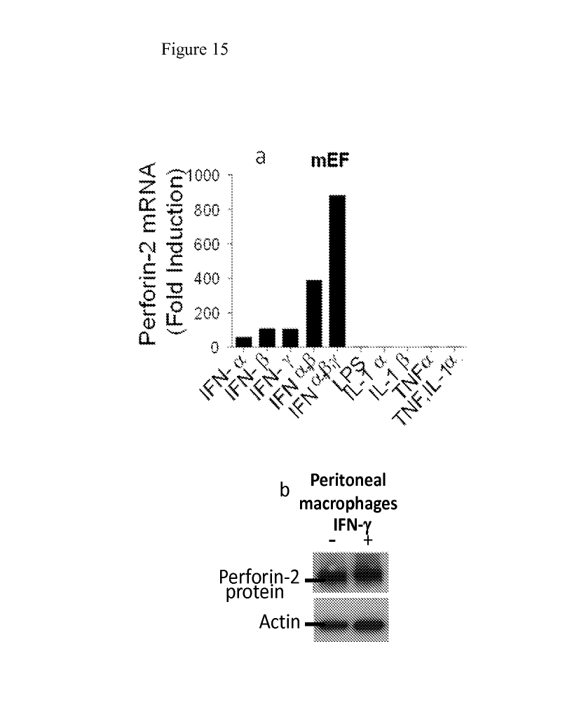

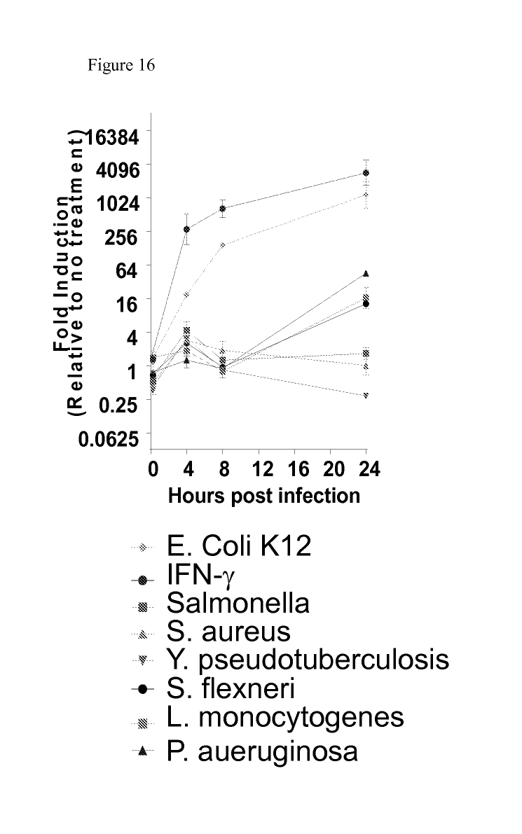

We examined 25 mouse and human cell lines and ex vivo cells to determine constitutive or inducible Perforin-2 expression by IFNα,β or γ or by intracellular bacterial infection. The results show that keratinocytes and phagocytic cells including PMN, macrophages and microglia express Perforin-2 constitutively. All non-phagocytic cells tested express Perforin-2 upon IFNα,β or γ induction or by intracellular infection (Table 2 and 3 and FIG. 15) (3). Bacteria that want to establish intracellular residence therefore must neutralize Perforin-2 to avoid being killed. We have previously shown that Chlamydiae actively suppress Perforin-2 induction in epithelial cells. We are in the process of identifying the Chlamydia genes responsible (4). FIG. 16 shows that many pathogenic bacteria including Salmonella typhimurium suppress Perforin-2 mRNA induction in MEF. Heat killed Salmonella and non-pathogenic E. coli on the other hand induce Perforin-2 to a similar degree as IFN-γ suggesting that suppression is an active process. EPEC and Yersinia pseudotuberculosis in addition use Cif (cycle inhibitory factor, (19, 20)) to suppress Perforin-2-killing (FIG. 5). How Mycobacteria neutralize Perforin-2 and/or suppress its expression is not known and is the overarching goal of this work.

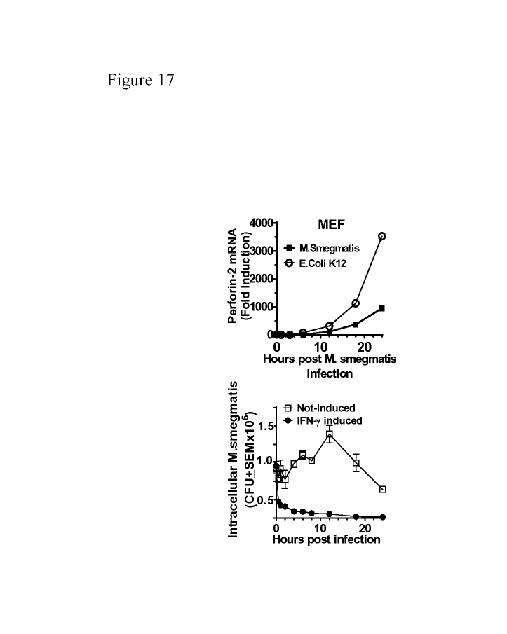

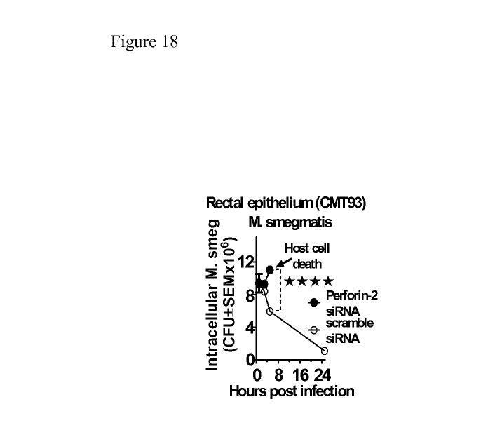

Intracellular infection of MEF with non-pathogenic E. coli induces high levels of Perforin-2 RNA (FIG. 16 and FIG. 17 upper panel). Intracellular M. smegmatis by comparison is a poor inducer of Perforin-2 compared to E. coli (FIG. 17). M. smegmatis replicate intracellularly for the first 12 hours after infection, prior to sufficient mRNA levels. Subsequently smegmatis is killed, coincident with increasing levels of Perforin-2 mRNA (FIG. 17, bottom panel, open squares). In contrast, if Perforin-2 is induced in MEF over night with IFN-γ then MEF instantly kill M. smegmatis during the first 10 hours (FIG. 17, bottom panel, filled circles). If Perforin-2 is knocked down with siRNA in IFN-γ induced epithelial cells (CMT93) M. smegmatis replicates and after 6 hours kills the host cell (FIG. 18). In the presence of Perforin-2 (scramble control) CMT93 require 24 h to completely kill M. smegmatis.

It is known that in addition to Perforin-2 upregulation, interferons induce hundreds of genes that are critical for innate and adaptive immune defense against infection, including the bactericidal gene iNOS for NO production (21, 22) and genes of the NOX family for ROS production (23). However our Perforin-2-knock-down data show conclusively that Perforin-2 is required for full bactericidal activity. We show additional support for this conclusion in cells in vitro in genetically Perforin-2 deficient (P-2^{-/-}) cells and in vivo in Perforin-2^{-/-} mice.

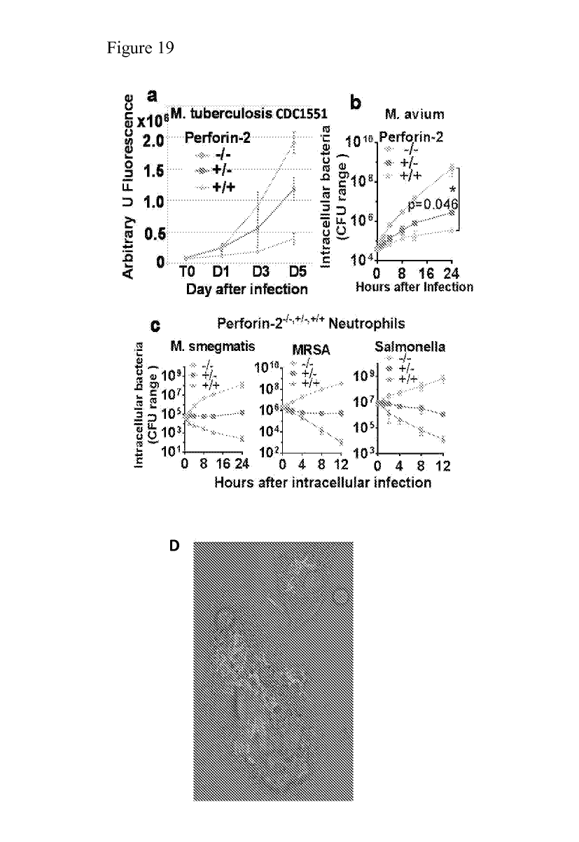

We have created Perforin-2 deficient mice and compared the bactericidal activity Perforin-2+/+, +/− and −/− macrophages and PMN for mycobacteria and other pathogenic bacteria. The data are illustrated in FIG. 19 show an extremely strong phenotype of Perforin-2−/− cells. M. tuberculosis (CDC1551) replicate significantly more rapidly in IFN-γ activated, Perforin-2−/− compared to +/+ bone marrow derived macrophages (p=0.0002, t-test), as measured with mCherry labeled bacteria (FIG. 19a). Similarly M. avium replicates significantly more rapidly in Perforin-2−/− than +/+PMN (p=0.046, t-test) (FIG. 19b). The data show that Perforin-2 strongly interferes with intracellular replication Mtb or M. avium. When Perforin-2 is overexpressed by transfection of RAW-macrophages, M. avium replication is completely stopped and the bacteria are killed (data not shown). A strong phenotype for Perforin-2 deficiency is also seen in FIG. 19c for M. smegmatis, MRSA USA300 (CL148, gift of Dr. L. Plano, U. Miami) and Salmonella typhimurium (RL144, gift of Dr. Galan, Yale). Our data clearly suggest that Mtb has potent mechanisms to attenuate Perforin-2 mediated killing. It is the overall goal to determine which step of Perforin-2 expression, localization or activity is inhibited by Mycobacterium tuberculosis (Mtb) and which of the mycobacterial genes are the primary Perforin-2 resistance and virulence genes.

### A. Suppression of Perforin-2 Induction by Mycobacteria

1. Elucidation of Host Pathways Relevant to Mtb-Mediated Inhibition of P-2 Expression in Non Phagocytic and Phagocytic Cells.

Experimental Design. Mycobacterium tuberculosis (Mtb) can infect and is found in the lung in both macrophages and non-phagocytic cells including epithelial cells, fibrocytes, adipocytes, and endothelial cells (24-26); mesenchymal stem cells may provide a niche (27). We will first establish how mycobacterial infection interferes with interferon- or microbial-mediated signal transduction pathways leading to Perforin-2 expression in MEF and in epithelial cells (CMT93). We will compare M. smegmatis, M. avium and Mtb at MOIs of 1 and 5. Mtb CDC1551 strain and tagged with smyc′::mCherry, smyc′::GFP and smyc′::ffluc have been used for analysis by plate reader, FACS caliber and confocal microscope. We will use confluent layers of non-phagocytic cells or macrophages in 24 well plates so that all bacteria will be phagocytosed, which will be verified by testing supernatants 12-16 hours after infection by plating and cfu. Samples for mRNA analysis will be taken provisionally at 0, 24 and 72 hours. Times will be altered as may be needed. Our readout for all of these approaches will be Perforin-2 qPCR of cDNA as a measure of P-2 message levels in whole-culture RNA samples. We will perform a series of control experiments in which mock or Mycobacteria infected cells are treated with recombinant IFNα, IFNβ, or IFNγ, combinations thereof, or heat killed controls. As control, we will examine expression of other host cell factors that respond to mycobacterial infection. For example, M. avium infection of macrophages reduces expression of IFN-γ inducible genes including Irf-1 and IFN-γRa and interferes with IFN-γ induced STAT1, JAk 1 and 2 phosphorylation (28). These experiments will establish whether Mtb interfere with a range of pathways and whether the effects are global or specific to Perforin-2. We will then test the temporal requirements for observed effects by treating with stimuli (e.g IFN) earlier in infection and asking whether Perforin-2 expression is still inhibited. We will also include antibiotic-induced blockage of de novo mycobacterial protein synthesis to establish whether and when in the infectious cycle Perforin-2 expression is inhibited.

We cannot exclude the possibility that Mycobacteria may have separate, but redundant factors that could inhibit Perforin-2 inducible expression via each pathway (upstream of type I or II-inducible transcription factors). We will begin by specifically examining potential roles of relevant transcription factors. We will use commercially available antibodies and activity tests to examine whether transcription factors like STATs, IRF1, 3, 4, and 7 are inhibited by mycobacterial infection with kinetics matching P-2 inhibition.

As a complementary approach, we will assess the requirements for Perforin-2 expression in non-phagocytic cells by constructing a Mycobacteria-responsive Perforin-2 reporter plasmid. The chromosomal Perforin-2 locus is open for transcription in more than 125 cells and cell lines as analyzed by DNAse hypersensitivity assays by the ENCODE project. About 4 kb upstream of transcription start is al DNAse I hypersensitivity cluster which is associated with 29 transcription factors identified by chromatin immunoprecipitation (CHIP) assays. The strongest signals in the Chip assay come from Pu.1, BATF, NFκB, Oct-2, POU2F2, PAX5, RXRA, BCL11, IRF4, TCF12, BCL3 and p300. These data suggest that the locus is open and ready to be transcribed rapidly upon appropriate signaling. This finding is consistent with data in table 2 and 3 indicating that virtually all cells can be rapidly induced by IFNs (and bacterial infection, FIG. 16) to transcribe Perforin-2. A 146111 by BAC construct containing the promoter and P-2 coding sequence has been created and expressed in eukaryotic cells (data not shown). We will begin by mobilization of the 4.5 kb region (spanning from ca 450 nt downstream to 4 kb upstream of the Perforin-2 start) into a promoter-less eukaryotic expression vector using PCR. The resulting construct can be easily manipulated via routine PCR-mediated cloning procedures. We will then replace the Perforin-2 coding sequence with a luciferase reporter construct to allow quantitative assessment of promoter activity. The resulting construct will be transfected into MEF cells and macrophages and we will confirm that the cloned region is subject to Mycobacteria-repressible expression in interferon-treated MEF cells and macrophages. Once these parameters are established, we will begin systematic deletion of predicted transcription factor binding sites to establish which factors contribute to Perforin-2 expression in epithelial cells and macrophages. We will prioritize removal of the DNAse hypersensitivity sites. If these are not involved, we will make a series of large deletions followed by smaller ones to narrow elements that are responsible for observed Perforin-2 expression patterns. To confirm the direct link between a respective DNA element and Mycobacteria-specific suppression of transcription we will infect with heat killed bacteria.

2. Does Mtb and Avium Suppress Already Induced Perforin-2?

This experiment will be carried out in two versions: (a) We will use RAW cells and bone marrow derived macrophages that express Perforin-2 protein constitutively, infect them with Mtb, M. smegmatis or M. avium (MOI 1, 5 and 10) and determine Perforin-2 protein expression in Western blots using commercial (Abcam) anti-peptide antibodies that detect denatured but not native Perforin-2. Time points will be from 0 to 72 h. (b) In a second version of the experiments, we will pre-induce Perforin-2 mRNA in MEF and macrophages by treatment over night with IFN-γ and then infect the cells with Mtb or other mycobacteria. Messenger RNA levels will be measured at multiple time points for up to 72 hours in parallel with assays for intracellular survival/replication using membrane impermeant antibiotics.

We will use confluent layers of cells in 24 well plates. At these low MOIs essentially all bacteria are phagocytosed precluding extracellular growth which will be verified by withdrawing and plating supernatants at 12 hours after infection. Results from the studies will depend on whether Mycobacteria infection directly blocks Perforin-2 expression at the promoter or globally interferes with signaling via the tested stimuli. A working model posits that Mycobacteria infection blocks Perforin-2 expression at a downstream event in signal transduction pathways, possibly a transcription factor or just upstream. Mycobacterial infections are sensitive to IFN-γ treatment which induces Perforin-2 transcription. This scenario suggests that mycobacteria could inhibit pathways upstream of IFN induction. Whether or not productive infection can block stimuli from heat-killed mycobacteria will be interesting and will shed light on whether viable mycobacteria interfere with sensing of pathogen associated molecular patterns (PAMPs). At the end of these experiments, we will know at what level Mycobacteria infection exerts an effect on Perforin-2-activating pathways.

### B. Elucidation of the Mycobacteria-Specific Factors Involved in Suppression of Perforin-2.

Experimental design. We will begin by replacing the luciferase gene in our Perforin-2 reporter construct with the eGFP coding sequence such that GFP is an indicator for Perforin-2 promoter activity. This reporter will be stably integrated into MEFs derived from Perforin-2 knockout mice (P-2−/− mice). In this way, we can directly examine Perforin-2 expression in the presence and absence of mycobacterial infection without interference from the bactericidal activity of Perforin-2. We will confirm that the reporter construct is responsive to mycobacterial infection and the stimuli found to be inhibited. This reporter system will then be used to identify Mtb mutants that are deficient in their ability to interfere with Perforin-2 expression.

### C. Determining Pathways Involved in Resistance to Perforin-2 Mediated Killing of Mtb

We will investigate the bacterial pathways involved in impacting susceptibility and resistance to Perforin-2-mediated killing of Mtb. A transposon insertion-site mapping method for genetic screening developed by Sassetti and Rubin (29, 30), known as TraSH, has proven to be an extremely effective approach for interrogating complex populations of Mtb mutants. The method enables the quantitative analysis of input and output mutant pools to detect those individual mutants enriched or depleted following selection. We have already used this method as a genetic approach for identifying metabolic pathways that are both positively and negatively selected for under different environmental conditions (1).

We will generate libraries of transposon-mutagenized Mtb containing approximately 200,000 independent insertions to ensure genome saturation. Perforin-2^{+/+} and ^{-/-} murine bone marrow-derived macrophages isolated from Perforin-2+/+ or ^{-/-} mice will be infected with pools of Mtb mutants at an MOI of either 1:1 or 5:1. In brief, approximately 2×10⁶ cfus from an aliquot of the input library will be used to infect wild-type and Perforin-2-deficient littermate. To limit the over-selection of fast growers, Mtb will be isolated at two time points, provisionally 24 hr and 72 hr. The control pool and the perforin-2-deficient pool of mutants will be isolated and both will be compared to the input pool in two biological replicates and two technical replicates, using TraSH. As detailed previously (1), genomic DNA from each pool will be partially digested with HinPI followed by MspI. 0.5-2 kb fragments will be purified and ligated to asymmetric adaptors, and transposon chromosome junctions amplified using PCR. We utilize a custom-designed, high-density microarray to identify the insertion sites. This array, synthesized by Agilent Technologies, consists of 60′ mer oligos every 350 by of the Mtb genome. We know from experience that this oligo density allows size-selected (200-500 bp), labeled probes to hybridize to at least one oligo and therefore provide sufficient coverage to identify the majority of insertion sites (1). Mutants that are significantly over-or under-represented in the output pools will be defined using the following criteria: arbitrary fluorescence intensity >300 in one of the channels, fluorescence ratio >3 and t test p value <0.05 (GeneSpring 12.5). The strength of this approach is that it provides a quantitative measure of selection through the relative abundance of different mutants enriched or depleted from the input pool. This allows one to “set” the degree of stringency to an appropriate level to reveal partial phenotypes. Similar data can be generated by RNASeq analysis but we find the microarray-based approach more cost-effective for analysis of multiple samples.

From this screen we will focus primarily on those mutants that are under-represented in the output pools and we expect to identify the following sets of mutants:

(1) Under-represented in Perforin-2+/+BMDM: Those bacteria impaired in intracellular survival through both Perforin-2 dependent and independent mechanisms.

(2) Over-represented in Perforin-2+/+BMDM: Those bacteria resistant to macrophage-mediate killing by both Perforin-2 dependent and independent mechanisms.

(3) Under-represented in Perforin-2−/− BMDM: Those bacteria impaired in intracellular survival through Perforin-2-independent mechanisms.

(4) Over-represented in Perforin-2−/− BMDM: Those bacteria resistant to macrophage-mediated killing through Perforin-2-independent mechanisms.

We have to use both Perforin-2−/− and +/+ litter mate macrophages to discriminate death from Perforin-2-dependent killing mechanisms from bacterial death due to mutation in unrelated pathways such as metabolic pathways required for intracellular survival, which would be common to both pools 1 and 3. Those mutants in classes 1 and 3 are the ones of greatest interest to us. Comparison of those mutants that are selected against in wild-type and Perforin-2^{-/-} BMDM should facilitate identification of mutants defective in those pathways that impair Perforin-2-dependent killing of Mtb, either at the transcriptional or functional level. Phenotypes will be validated by the generation of clean knockouts and through complementation of genes of interest as published (16).

Many genetic screens work best on single gene/single function, which would be the case if a phenotype were due to a single secreted effector. This is less true for TraSH analysis because we are able to quantify the negative or positive selection on multiple genetic loci simultaneously. This does require more analysis but we would argue that the TraSH approach should allow identification of multi-loci phenotypes, or pathways. For example; macrophage behavior is known to be influenced by bacterial cell wall lipids (31, 32). These lipids are the products of multiple genes therefore if mutants defective in the synthesis of such mediators are selected against we should be able to identify several genes in the synthetic pathway.

One additional concern is complementation in trans. If the altered macrophage phenotype is induced by bacterial cell wall lipids it is feasible that all cells in the culture will be affected. This would negate the screen. However, if this is the case we can, as we have done previously (31-33), treat the mice or macrophages with isolated mycobacterial lipids and assay whether this impact the ability of the cells to kill an unrelated pathogen, such as Salmonella or Chlamydia.

Mtb will be mutagenized and candidates will be identified by Perforin-2+/+ and −/− selection in macrophages using the TraSH approach as described. The genes that confer resistance to Perforin-2-mediated killing will be validated by the generation of clean knockouts and through complementation of genes of interest as published (16). We will identify the step in Perforin-2 expression, activation or killing that is inhibited by the identified. It is possible that a Perforin-2 resistance gene does not directly affect Perforin-2 but mediated Perforin-2-resistance, for instance via its role on genes affecting bacterial envelop and repair of Perforin-2 damage. We found that M. smegmatis were able to repair some Perforin-2 damage to the envelop if lysozyme was absent but not in its presence (3).

### II. Does Mtb Inhibit Translocation to the Bacterium Containing Vacuole?

A. Structure of Perforin-2 and Mechanism of Activation.

Perforin-2, encoded by MPEG-1 (5), is an integral transmembrane protein containing a N-terminal Membrane Attack Complex Perforin domain (MACPF) connected via a novel domain, designated P2 by us, to the transmembrane domain and a C-terminal short (38AA) cytoplasmic domain (FIG. 2). The MACPF polymerization and killing domain is located inside membrane vesicles in the cytosol (FIG. 2). Perforin-2 is highly conserved down to sponges including the MACPF and P2 domains (3, 34). The cytoplasmic domain is conserved among vertebrates and in mammals as indicated in FIG. 2 suggesting conserved signaling elements. The function of Perforin-2 was not known until our publication that demonstrated its bactericidal activity (3, 4). We introduced a Y to F mutation (red arrow, FIG. 2) which inactivated Perforin-2 mediated killing of intracellular bacteria but not expression (data not shown), suggesting functional importance of the cytoplasmic domain. The MACPF domain is also found in the pore-forming proteins of complement, including pore-forming poly-C9, and in poly-Perforin-1 (8, 9, 35, 36). We determined whether Perforin-2 via its MACPF can form membrane/cell wall pores. The pore-forming MACPF killer domain is located in the vesicle lumen (FIG. 2) suggesting that it could form pores on targets (bacteria) enclosed by the membrane. In FIG. 1, M. smegmatis (middle) and MRSA (right panel) were isolated form IFN-γ induced MEF 5 hours after infection, the bacteria disrupted by polytron and the cell walls examined by negative staining electron-microscopy (FIG. 1, 150,000 fold magnification). The left panel shows poly-Perforin-2 in eukaryotic phospholipid bilayer membranes. The bacterial cell walls bear clusters of connected pores of ˜Å100 diameter, similar in size to poly-C9 pores of complement. Control cell walls have no such pores (not shown). Pores are not detected when Perforin-2 is knocked down with siRNA and bacteria are not killed (not shown). The pictures indicate that Perforin-2 is a pore-forming protein and that clustered pores are present on bacterial cell walls isolated from Perforin-2 expressing, bactericidal MEF. The surface area of the M. smegmatis fragment attacked and clustered with Perforin-2-polymers in FIG. 1, panel b, is >0.16 μm² large and represents more than 6% of the total surface area. Similar damage is seen also on MRSA (FIG. 1, panel c). Such extensive cell wall damage is likely to considerably impair the normal protective function of the bacterial envelop and provide access for chemical attack by ROS, NO and hydrolases including lysozyme.

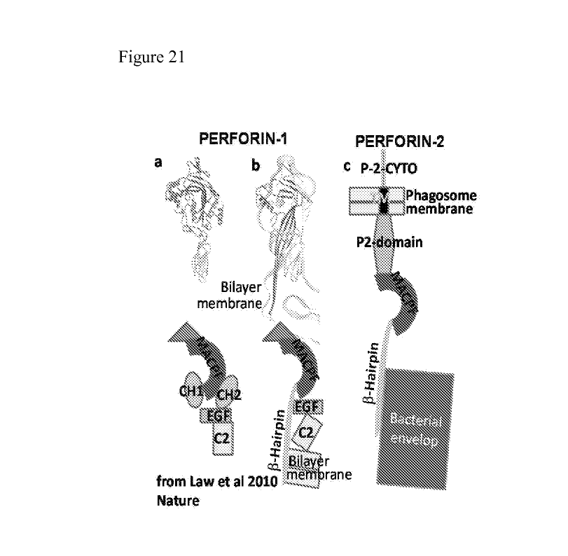

The refolding of CH1 and CH2 of the MACPF domain during polymerization, membrane insertion and attack has recently been elucidated by crystallization in combination with cryo-electron-microscopy (2) and confirms our original model (37). In FIG. 21 we model the molecular mechanism of Perforin-2 attached to the phagosome membrane attacking a bacterium inside the phagosome. According to this model the MACPF domain of Perforin-2 damages the outer layer of the envelop (FIG. 21c) of a bacterium trapped in the phagosome.

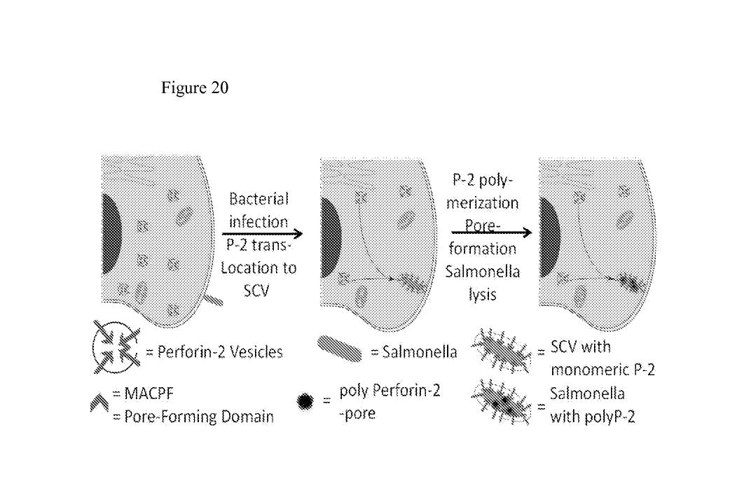

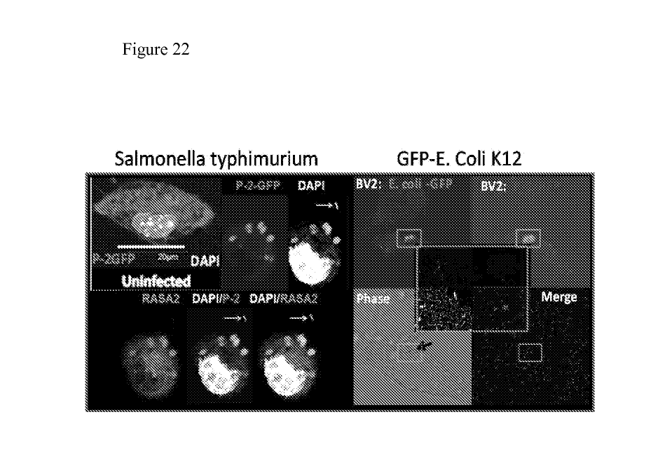

The presence of the membrane protein Perforin-2 in membrane vesicles stored throughout the cytoplasm (FIG. 22, upper left) requires translocation to the bacterium containing vacuole upon intracellular infection, which is modeled in FIG. 20. Once fused with endosome/vacuole membrane Perforin-2 is triggered to polymerize and attack and kill the bacterium inside the endosome/vacuole. Confocal studies shown in FIG. 22 appear to support this model. In the left panel, upper left, is an uninfected microglia BV2 cell transfected with Perforin-2-GFP (green) and stained with DAPI, white, shown in false color for better visibility. The other panels show Perforin-2-GFP transfected BV-2 infected with Salmonella (MOI 30), fixed after 5 minutes and stained with anti RASA2/GAP1M antibody (orange). Endogenous Perforin-2 is knocked down with 3′UTR specific siRNA. The arrow depicts an intact Salmonella rod outside the cells stained with DAPI. The green, white and orange egg shaped structures inside the cell are endosomes that appear to contain Salmonellae that have released their DNA due to Perforin2 attack. The merged images indicate colocalization. Right panel, FIG. 22: GFP-marked E. coli in Perforin-2-RFP transfected BV2 fixed 5 min after infection. The bacterium containing endosome is zoomed in the center panels and shows the bacterium in the endosome phase (lower left). The green GFP (upper left) shows the bacterium fragmented and partly leaked out of the bacterium. Perforin-2-RFP (upper right) is highly concentrated on the endosome membrane and the bacterial surface. The merged image indicates colocalization.

As may be expected for an entirely novel pathway, many details of Perforin-2 activation, targeting to the invading bacterium and killing are still unknown. However, we have identified several Perforin-2 activating proteins (Table 1) and collected evidence that allows the construction of a model for Perforin-2 activation and attack of bacteria inside endocytic vacuoles as shown in FIGS. 20 and 4.

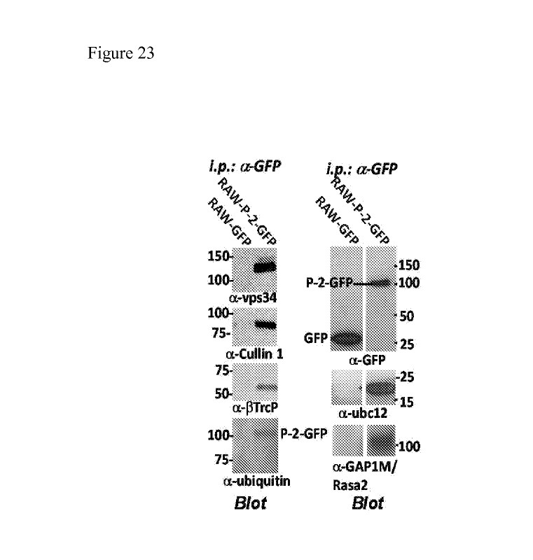

Experimental design: Perforin-2 function and potential interruption of its function by bacterial factors will be monitored in Perforin-2-coimmunoprecipitation assays. Perforin-2 interacts with vps34, RASA2/GAP1M, ubc12, cullin-1 and βTrcP in IFN-γ and LPS activated RAW cells (FIG. 23, 4). Perforin-2 is mono-ubiquitylated which is often used as trafficking signal. Interaction of Perforin-2 with its interacting proteins is necessary for the function of Perforin-2 translocation to the bacterium containing vacuole and/or for triggering Perforin-2 polymerization and killing of intracellular bacteria. Knock down of the interacting proteins with siRNA blocks or greatly inhibits the killing activity of Perforin-2 (data not shown). Likewise, interference by bacterial factors would protect bacteria from being killed. Interference of interaction could be direct or it could be by inhibition of earlier activation steps. For instance the cytoplasmic domain of Perforin-2 has 1 conserved Y and 2 conserved S-phosphorylation sites (FIG. 2). We suggest that bacterial infection and endocytosis triggers Ca-fluxes and unknown kinases to phosphorylate (or phosphatases to dephosphorylate) Perforin-2-cyto as one of the earliest steps to initiate translocation of Perforin-2. Translocation probably requires interaction with vps34 and RASA2/GAP1M. Vps34 is in complex with vps15 a kinase that requires activation. Interference of bacteria with the early activation steps could prevent subsequent interaction of these putative translocation proteins with Perforin-2. Perforin-2 function upon infection with mycobacteria will also be monitored by confocal microscopy as shown in FIG. 22. This assay may be able to distinguish between translocation and polymerization. It is possible that bacteria do not interfere with translocation but inhibit Perforin-2 polymerization. In that case the labeled bacteria would be seen inside the endosomal vacuole but they would not be killed, e.g. would not release their DNA or become fragmented as seen in FIG. 22.

FIG. 4 shows our model of Perforin-2 in the membrane of a Mtb containing vacuole with the Perforin-2-cyto associated interacting proteins that control function. FIG. 5 shows the model for Perforin-2 polymerization based on the interaction of Perforin-2-cyto with ubc12, Cullin-1 and βTrcP all of which are required to assemble the Cullin-Ring-Ubiquitin-Ligase that is required for Perforin-2 function (FIG. 5). We suggest ubiquitylation of the lysine cluster (FIG. 2) of Perforin-2-cyto is the signal for proteasome mediated degradation of the cytoplasmic domain resulting in polymerization. This proteolytic cleavage is distantly analogous to complement in which the proteolytic cleavage of C5 to C5b is the trigger for the assembly of the membrane attack complex and polymerization of C9. C6, C7, C8 and C9 all have MACPF domains that copolymerize with 14-16 C9 molecules, poly C9 forming, the pore/hole of 100 Å (38).

B. Phosphorylation and Coimmunoprecipitation.

Bone marrow derived and IFN-γ activated macrophages or RAW-cells will be transiently transfected with Perforin-2-GFP and infected with mCherry-mycobacteria at MOIs from 1 to 10. Samples will be taken at early times provisionally from 2 min up to 72 h. Times will be adjusted according to the experience collected. Analysis will be done by Perforin-2 coimmunoprecipitation of the proteins indicated in FIG. 23 and table 1. We will compare M. smegmatis, M. avium and confirm with Mtb; among these three mycobacterial species M. smegmatis will serve as positive control since it can be killed relatively efficiently by Perforin-2. Another positive control will be E. coli K12 which is non-pathogenic and has no known resistance genes or plasmids. We will also look for kinase action. The putative kinases phosphorylating Y and S in Perforin-2-cyto are not known, but candidates (Tec and Nek) are predicted by algorhythms. We will blot Perforin-2 immunoprecipitates with anti-phospho-tyrosine and anti-phospho-serine antibodies prior to and after different times of infection.

Our current data suggest that Perforin-2 mediated killing proceeds in a cascade of three synchronized steps. (1) Kinase (phosphatase) activation: The conserved phosphorylation sites on Perforin-2-cyto suggest kinase activation most likely as the first step after bacterial attachment and endocytosis/phagocytosis. (2) Translocation: Perforin-2 loaded membrane vesicles are translocated from the cytosol to and fuse with the bacterium containing endosome/phagosome membrane. (3) Polymerization: Perforin-2-polymerization needs to be triggered and timed at exactly the correct moment when the bacterium inside the endosome comes close to the endosome membrane and touches the N-terminal MACPF-domain of Perforin-2. At that time polymerization is triggered and a chain reaction of polymerization hits the bacterial surface and forms clustered pores in that area of the bacterial surface that is in close enough proximity to the MACPF. Membrane damage facilitates the bactericidal action of ROS, NO and lysozyme (3).

Inhibition or alteration of the kinase (or phosphatase) steps will be followed over time with anti-phospho-antibodies or P32 labeling to reveal the effects of Mtb and M. avium that are different from the positive controls E. coli and M. smegmatis. Blockade at that early level is expected to also block translocation and polymerization and killing. It is possible that Mycobacteria prematurely trigger polymerization prior to translocation. Poly-Perforin-2 is expected to be killing-inactive as are poly-C9 and poly-Perforin-1.

Vps34 and RASA2/GAP1M (and additional proteins not yet identified) are the likely candidates required for translocation. If their interaction with Perforin-2 is hampered by Mycobacterial factors translocation will be inhibited which we will confirm by confocal microscopy. To counteract the bacterial inhibition we will overexpress vps34 and/or RASA2/GAP1M to restore killing activity. Mtb is known to interfere with vps34 via ManLam and Ca²⁺ mobilization. The SapM phosphatase may dephosphorylate PI3P (39-44). Perforin-2-cyto interacts and coimmunoprecipitates with both the PI3-kinase vps34 and PI3P binding protein RASA2/GAP1M. Interference at this level clearly would have strong negative effects on Perforin-2 function.

C. Polymerization.

Bacterial killing requires Perforin-2 polymerization and physical damage to the bacterial surface. Bacterial death therefore can be taken as indirect evidence that polymerization has occurred including all the other earlier steps for Perforin-2 activation. Our data suggest that polymerization is triggered by ubiquitination of Perforin-2-cyto at the lysine cluster by a Cullin-Ring-ubiquitin-Ligase (CRL). Perforin-2 coimmunoprecipitates and Perforin-2-cyto interacts in the yeast two hybrid system with ubc12, the principal NEDD8 ligase required for CRLs (45, 46). Perforin-2 also coimmunoprecipitates with the cullin1 scaffolding protein which is the NEDD8-substrate and with βTrcP which is the Fbox protein associated with cullin1 and Skp1 recognizing Perforin-2-cyto (FIG. 23). Finally, Perforin-2 immunoprecipitates are ubiquitinated.

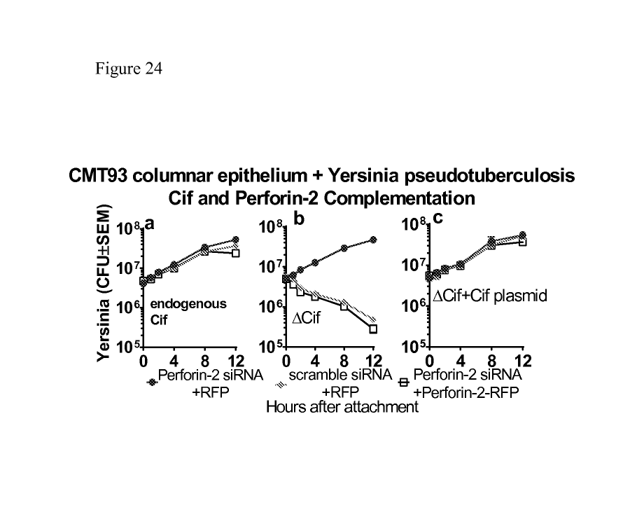

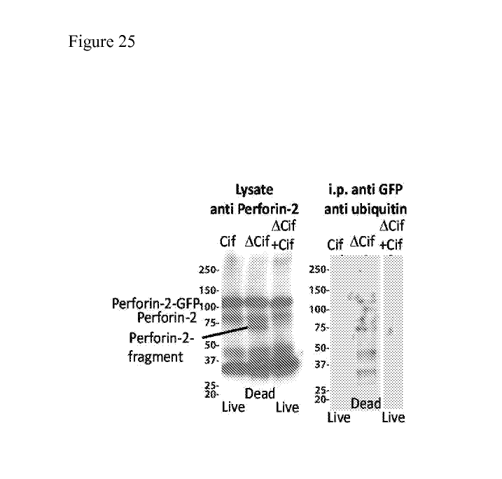

Further support for the requirement of a CRL derives from our finding that the Cif-plasmid, known to inactivate NEDD8 (FIG. 5) (19, 20), blocks Perforin-2 mediated killing of Cif containing Yersinia pseudotuberculosis. Cif deficient Yersinia in contrast are sensitive to Perforin-2 killing by endogenous Perforin-2 or by complemented Perforin-2-GFP (FIG. 24). Lysates of killed Yersinia blotted with anti-Perforin-2 show a new Perforin-2-fragment band not detected when Cif is present and the bacteria survive. The finding suggests Perforin-2 cleavage as a consequence of activation. Moreover, Perforin-2-GFP immunoprecipitates (with anti GFP) are ubiquitin-negative when killing is blocked by Cif and ubiquitin positive when Cif is absent and the bacteria are killed (FIG. 25). The data suggest that ubiquitination and cleavage of Perforin-2-cyto-GFP may be necessary for Perforin-2 polymerization and killing of intracellular bacteria. The ubiquitination and Perforin-2-cleavage assay therefore will be developed as a (non-quantitative) surrogate assay for Perforin-2-polymerization.

There are no assays available for measuring polymerization of Perforin-2 directly, which is also true for Perforin-1 and poly-C9. Killing implies polymerization and can be used to indicate that polymerization has taken place. As discussed above, our data indicates that the final step is induction of Perforin-2 polymerization in the endosome by ubiquitylation of the cytoplasmic domain and cleavage/degradation by the proteasome (FIG. 4). The evidence in FIG. 25 and FIG. 23 supports this. Further support comes from the potent Perforin-2 blocking activity of Cif (FIG. 24) which completely protects Y. pseudotuberculosis from Perforin-2 killing via blocking NEDD8 which is required for CRL mediated ubiquitylation of Perforin-2. Salmonella typhimurium encodes a deubiquitinase, SseL, which has been linked autophagy (47). It is possible that SseL also is a Perforin-2 resistance factor. We have evidence that bacterial killing by autophagy also requires Perforin-2. CYLD is a cell based deubiquitinase that down regulates inflammation. Expression of CYLD is relatively low under physiological conditions but is significantly upregulated upon bacterial infections in respiratory systems (48-51); upregulation of CYLD by bacteria is achieved through inhibition of phosphodiesterase 4B (52). Increased CYLD levels inhibit NFκB activation and may also deubiquitinate Perforin-2, thereby blocking polymerization and killing. We will therefore use deubiquitinase inhibitors and siRNA to determine efficiency of Perforin-2 dependent Mtb and M. avium killing.

III. Importance of Perforin-2 in Controlling Mycobacteria In Vivo

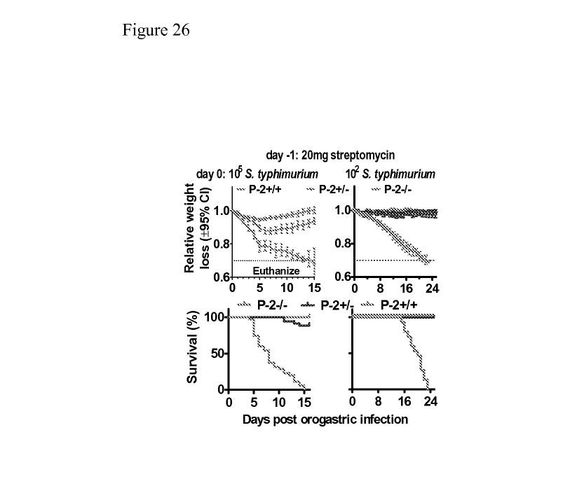

We have created Perforin-2 deficient mice by homologous gene replacement. As shown in FIG. 19 Mtb and M. avium replicate significantly more rapid in Perforin-2 deficient PMN and BMDM that in Perforin-+/+ cells. These data strongly suggest that Perforin-2 is important to restrain intracellular mycobacterial replication, at least in vitro. In vivo challenge of Perforin-2−/−, +/−, and +/+ litter mates by orogastric infection with Salmonella typhimurium RL144 and by epicutaneous infection with MRSA CL1380 revealed a strong phenotype. Perforin-2−/− mice die from Salmonella challenge that is cleared by +/+ and Perforin-2+/− litter mates (FIG. 26). Similar lethality in Perforin−/− but not +/− or +/+ mice is observed by epicutaneous MRSA infection (data not shown). The data indicate that Perforin-2 is a critical effector for anti-bacterial defense in vivo. In the absence of Perforin-2 pathogenic bacteria rapidly disseminate systemically, create bacteremia and replicate to 10³ to 10⁴ fold higher levels in spleen liver and kidneys than in Perforin-2+/+ mice. We predict, based on the in vitro data in FIG. 19a, b that Perforin-2 is also a critical effector in vivo against and Mtb and M. avium and that Perforin-2−/− mice will succumb much more quickly and to lower doses of infection than +/+ or +/− littermates.

Experimental plan: We will infect Perforin-2−/−, +/− and +/+ litter mates by the intranasal route and by i.p. injection with mCherry-Mtb. Graded doses will be used for infection to determine the level of defense in the presence of 2, 1 or no allele of Perforin-2. We will create Mtb mutants deficient in identified Perforin-2 resistance genes and use them for in vivo challenge of Perforin-2−/−+/− and +/+ litter mates. Groups of 12 mice will be used and 4 infectious dose levels of bacteria will be used for each experiment. Certified BSL3 animal facilities will be used. The mice will be followed by weight and by clinical observation for behavior and well-being. Anti-inflammatory drugs and pain medicine will be administered as needed upon consultation with our veterinarians in the Division of veterinary Research. Groups of 3 mice will be sacrificed at 4-6 weeks intervals or earlier if moribund. Necropsy will include histopathological analysis of lungs, liver, spleen and the intestinal tract. In addition samples from these organs will be used to determine CFU. Tissues from mice challenged with mCherry-Mtb and its deletion mutants will also be analyzed flow cytometry and fluorescence microscopy.

Perforin-2 deficient mice kept in pathogen free barrier facilities have no pathologic phenotype. The normal commensal gut and skin flora does not require Perforin-2. Pathogenic bacteria, including Mycobacteria are invasive in vivo and require active defense by Perforin-2. We predict that Perforin-2−/− will be significantly more susceptible to Mtb than w.t. mice. Clinically this will appear as rapid weight loss and as rapid dissemination of Mtb to multiple organs. The clinical picture may resemble miliary tuberculosis, a form of disseminated hyperacute tuberculosis seen in patients and in children which is rapidly lethal if untreated. Using Mtb mutants in which Perforin-2 resistance genes have been deleted are expected to be less pathogenic in Perforin-2+/+ and +/− mice but may remain equally pathogenic in Perforin-2−/− mice. Screening the various deletion mutants of Mtb in this in vivo system will give us important insights into the critical components of Mtb that resist Perforin-2-dependent killing and provide Mtb with virulence. These insights will also help to determine which step of the Perforin-2 activation pathway is inhibited. And it will allow us to develop biological or small molecular drugs to counteract the Mtb resistance pathway and enable Perforin-2 to destroy the bacillus.

### REFERENCES

1. Lee, W., B. C. VanderVen, R. J. Fahey, and D. G. Russell. 2013. Intracellular Mycobacterium tuberculosis exploits host-derived fatty acids to limit metabolic stress. The Journal of biological chemistry 288: 6788-6800.
2. Law, R. H., N. Lukoyanova, I. Voskoboinik, T. T. Caradoc-Davies, K. Baran, M. A. Dunstone, M. E. D'Angelo, E. V. Orlova, F. Coulibaly, S. Verschoor, K. A. Browne, A. Ciccone, M. J. Kuiper, P. I. Bird, J. A. Trapani, H. R. Saibil, and J. C. Whisstock. 2010. The structural basis for membrane binding and pore formation by lymphocyte perforin. Nature 468: 447-451.
3. McCormack, R., L. R. de Armas, M. Shiratsuchi, J. E. Ramos, and E. R. Podack. 2013 Inhibition of intracellular bacterial replication in fibroblasts is dependent on the perforin-like protein (perforin-2) encoded by macrophage-expressed gene 1. Journal of innate immunity 5: 185-194.
4. Fields, K. A., R. McCormack, L. R. de Armas, and E. R. Podack. 2013. Perforin-2 Restricts Growth of Chlamydia trachomatis in Macrophages. Infect Immun 81: 3045-3054.
5. Spilsbury, K., M. A. O'Mara, W. M. Wu, P. B. Rowe, G. Symonds, and Y. Takayama. 1995. Isolation of a novel macrophage-specific gene by differential cDNA analysis. Blood 85: 1620-1629.
6. He, X., Y. Zhang, and Z. Yu. 2011. An Mpeg (macrophage expressed gene) from the Pacific oyster Crassostrea gigas: molecular characterization and gene expression. Fish Shellfish Immunol 30: 870-876.
7. Kemp, I. K., and V. E. Coyne. 2011. Identification and characterisation of the Mpeg1 homologue in the South African abalone, Haliotis midae. Fish Shellfish Immunol 31: 754-764.
8. Podack, E. R., and G. Dennert. 1983. Assembly of two types of tubules with putative cytolytic function by cloned natural killer cells. Nature 302: 442-445.
9. Podack, E. R., and J. Tschopp. 1982. Circular polymerization of the ninth component of complement. Ring closure of the tubular complex confers resistance to detergent dissociation and to proteolytic degradation. The Journal of biological chemistry 257: 15204-15212.
10. Tan, S., N. Sukumar, R. B. Abramovitch, T. Parish, and D. G. Russell. 2013. Mycobacterium tuberculosis responds to chloride and pH as synergistic cues to the immune status of its host cell. PLoS Pathog 9: e1003282.
11. Russell, D. G. 2013. The evolutionary pressures that have molded Mycobacterium tuberculosis into an infectious adjuvant. Curr Opin Microbiol 16: 78-84.
12. Rohde, K. H., D. F. Veiga, S. Caldwell, G. Balazsi, and D. G. Russell. 2012. Linking the transcriptional profiles and the physiological states of Mycobacterium tuberculosis during an extended intracellular infection. PLoS Pathog 8: e1002769.
13. Podinovskaia, M., W. Lee, S. Caldwell, and D. G. Russell. 2013. Infection of macrophages with Mycobacterium tuberculosis induces global modifications to phagosomal function. Cell Microbiol 15: 843-859.
14. Pethe, K., D. L. Swenson, S. Alonso, J. Anderson, C. Wang, and D. G. Russell. 2004. Isolation of Mycobacterium tuberculosis mutants defective in the arrest of phagosome maturation. Proc Natl Acad Sci USA 101: 13642-13647.
15. McKinney, J. D., K. Honer zu Bentrup, E. J. Munoz-Elias, A. Miczak, B. Chen, W. T. Chan, D. Swenson, J. C. Sacchettini, W. R. Jacobs, Jr., and D. G. Russell. 2000. Persistence of Mycobacterium tuberculosis in macrophages and mice requires the glyoxylate shunt enzyme isocitrate lyase. Nature 406: 735-738.
16. Abramovitch, R. B., K. H. Rohde, F. F. Hsu, and D. G. Russell. 2011. aprABC: a Mycobacterium tuberculosis complex-specific locus that modulates pH-driven adaptation to the macrophage phagosome. Mol Microbiol 80: 678-694.
17. Podack, E. R., H. Hengartner, and M. G. Lichtenheld. 1991. A central role of perforin in cytolysis? Annual review of immunology 9: 129-157.
18. Podack, E. R., and A. Kupfer. 1991. T-cell effector functions: mechanisms for delivery of cytotoxicity and help. Annual review of cell biology 7: 479-504.
19. Cui, J., Q. Yao, S. Li, X. Ding, Q. Lu, H. Mao, L. Liu, N. Zheng, S. Chen, and F. Shao. 2010. Glutamine deamidation and dysfunction of ubiquitin/NEDD8 induced by a bacterial effector family. Science 329: 1215-1218.
20. Taieb, F., J. P. Nougayrede, and E. Oswald. 2011. Cycle inhibiting factors (cifs): cyclomodulins that usurp the ubiquitin-dependent degradation pathway of host cells. Toxins 3: 356-368.
21. Xie, Q. W., R. Whisnant, and C. Nathan. 1993. Promoter of the mouse gene encoding calcium-independent nitric oxide synthase confers inducibility by interferon gamma and bacterial lipopolysaccharide. J Exp Med 177: 1779-1784.
22. Brandonisio, O., M. A. Panaro, M. Sisto, A. Acquafredda, L. Fumarola, D. Leogrande, and V. Mitolo. 2001. Nitric oxide production by Leishmania-infected macrophages and modulation by cytokines and prostaglandins. Parassitologia 43 Suppl 1: 1-6.
23. Rada, B., and T. L. Leto. 2008. Oxidative innate immune defenses by Nox/Duox family NADPH oxidases. Contributions to microbiology 15: 164-187.
24. Gomez, J. E., and J. D. McKinney. 2004. M. tuberculosis persistence, latency, and drug tolerance. Tuberculosis 84: 29-44.
25. Hernandez-Pando, R., M. Jeyanathan, G. Mengistu, D. Aguilar, H. Orozco, M. Harboe, G. A. Rook, and G. Bjune. 2000. Persistence of DNA from Mycobacterium tuberculosis in superficially normal lung tissue during latent infection. Lancet 356: 2133-2138.
26. Neyrolles, O., R. Hernandez-Pando, F. Pietri-Rouxel, P. Fornes, L. Tailleux, J. A. Barrios Payan, E. Pivert, Y. Bordat, D. Aguilar, M. C. Prevost, C. Petit, and B. Gicquel. 2006. Is adipose tissue a place for Mycobacterium tuberculosis persistence? PLoS One 1: e43.
27. Das, B., S. S. Kashino, I. Pulu, D. Kalita, V. Swami, H. Yeger, D. W. Felsher, and A. Campos-Neto. 2013. CD271(+) bone marrow mesenchymal stem cells may provide a niche for dormant Mycobacterium tuberculosis. Science translational medicine 5: 170ra113.
28. Hussain, S., B. S. Zwilling, and W. P. Lafuse. 1999. Mycobacterium avium infection of mouse macrophages inhibits IFN-gamma Janus kinase-STAT signaling and gene induction by down-regulation of the IFN-gamma receptor. J Immunol 163: 2041-2048.
29. Sassetti, C. M., D. H. Boyd, and E. J. Rubin. 2001. Comprehensive identification of conditionally essential genes in mycobacteria. Proc Natl Acad Sci USA 98: 12712-12717.
30. Sassetti, C. M., and E. J. Rubin. 2003. Genetic requirements for mycobacterial survival during infection. Proc Natl Acad Sci USA 100: 12989-12994.
31. Bowdish, D. M., K. Sakamoto, M. J. Kim, M. Kroos, S. Mukhopadhyay, C. A. Leifer, K. Tryggvason, S. Gordon, and D. G. Russell. 2009. MARCO, TLR2, and CD14 are required for macrophage cytokine responses to mycobacterial trehalose dimycolate and Mycobacterium tuberculosis. PLoS Pathog 5: e1000474.
32. Geisel, R. E., K. Sakamoto, D. G. Russell, and E. R. Rhoades. 2005. In vivo activity of released cell wall lipids of Mycobacterium bovis bacillus Calmette-Guerin is due principally to trehalose mycolates. J Immunol 174: 5007-5015.
33. Rhoades, E. R., R. E. Geisel, B. A. Butcher, S. McDonough, and D. G. Russell. 2005. Cell wall lipids from Mycobacterium bovis BCG are inflammatory when inoculated within a gel matrix: characterization of a new model of the granulomatous response to mycobacterial components. Tuberculosis 85: 159-176.
34. D'Angelo, M. E., M. A. Dunstone, J. C. Whisstock, J. A. Trapani, and P. I. Bird. 2012. Perforin evolved from a gene duplication of MPEG1, followed by a complex pattern of gene gain and loss within Euteleostomi. BMC Evol Biol 12: 59.
35. DiScipio, R. G., M. R. Gehring, E. R. Podack, C. C. Kan, T. E. Hugli, and G. H.

Fey. 1984. Nucleotide sequence of cDNA and derived amino acid sequence of human complement component C9. Proc Natl Acad Sci USA 81: 7298-7302.
36. Tschopp, J., H. J. Muller-Eberhard, and E. R. Podack. 1982. Formation of transmembrane tubules by spontaneous polymerization of the hydrophilic complement protein C9. Nature 298: 534-538.
37. Tschopp, J., E. R. Podack, and H. J. Muller-Eberhard. 1982. Ultrastructure of the membrane attack complex of complement: detection of the tetramolecular C9-polymerizing complex C5b-8. Proc Natl Acad Sci USA 79: 7474-7478.
38. Podack, E. R. 1984. Molecular composition of the tubular structure of the membrane attack complex of complement. The Journal of biological chemistry 259: 8641-8647.
39. Via, L. E., D. Deretic, R. J. Ulmer, N. S. Hibler, L. A. Huber, and V. Deretic. 1997. Arrest of mycobacterial phagosome maturation is caused by a block in vesicle fusion between stages controlled by rab5 and rab7. The Journal of biological chemistry 272: 13326-13331.
40. Philips, J. A. 2008. Mycobacterial manipulation of vacuolar sorting. Cell Microbiol 10: 2408-2415.
41. Fratti, R. A., J. M. Backer, J. Gruenberg, S. Corvera, and V. Deretic. 2001. Role of phosphatidylinositol 3-kinase and Rab5 effectors in phagosomal biogenesis and mycobacterial phagosome maturation arrest. The Journal of cell biology 154: 631-644.
42. Vieira, O. V., R. E. Harrison, C. C. Scott, H. Stenmark, D. Alexander, J. Liu, J. Gruenberg, A. D. Schreiber, and S. Grinstein. 2004. Acquisition of Hrs, an essential component of phagosomal maturation, is impaired by mycobacteria. Molecular and cellular biology 24: 4593-4604.
43. Vergne, I., J. Chua, H. H. Lee, M. Lucas, J. Belisle, and V. Deretic. 2005. Mechanism of phagolysosome biogenesis block by viable Mycobacterium tuberculosis. Proc Natl Acad Sci USA 102: 4033-4038.
44. Alix, E., S. Mukherjee, and C. R. Roy. 2011. Subversion of membrane transport pathways by vacuolar pathogens. The Journal of cell biology 195: 943-952.
45. Merlet, J., J. Burger, J. E. Gomes, and L. Pintard. 2009. Regulation of cullin-RING E3 ubiquitin-ligases by neddylation and dimerization. Cellular and molecular life sciences: CMLS 66: 1924-1938.
46. Petroski, M. D., and R. J. Deshaies. 2005. Function and regulation of cullin-RING ubiquitin ligases. Nature reviews. Molecular cell biology 6: 9-20.
47. Mesquita, F. S., M. Thomas, M. Sachse, A. J. Santos, R. Figueira, and D. W. Holden. 2012. The Salmonella deubiquitinase SseL inhibits selective autophagy of cytosolic aggregates. PLoS Pathog 8: e1002743.
48. Trompouki, E., E. Hatzivassiliou, T. Tsichritzis, H. Farmer, A. Ashworth, and G. Mosialos. 2003. CYLD is a deubiquitinating enzyme that negatively regulates NF-kappaB activation by TNFR family members. Nature 424: 793-796.
49. Jono, H., J. H. Lim, L. F. Chen, H. Xu, E. Trompouki, Z. K. Pan, G. Mosialos, and J. D. Li. 2004. NF-kappaB is essential for induction of CYLD, the negative regulator of NF-kappaB: evidence for a novel inducible autoregulatory feedback pathway. The Journal of biological chemistry 279: 36171-36174.
50. Yoshida, H., H. Jono, H. Kai, and J. D. Li. 2005. The tumor suppressor cylindromatosis (CYLD) acts as a negative regulator for toll-like receptor 2 signaling via negative cross-talk with TRAF6 AND TRAF7. The Journal of biological chemistry 280: 41111-41121.
51. Espinosa, L., S. Cathelin, T. D'Altri, T. Trimarchi, A. Statnikov, J. Guiu, V. Rodilla, J. Ingles-Esteve, J. Nomdedeu, B. Bellosillo, C. Besses, O. Abdel-Wahab, N. Kucine, S. C. Sun, G. Song, C. C. Mullighan, R. L. Levine, K. Rajewsky, I. Aifantis, and A. Bigas. 2010. The Notch/Hes1 pathway sustains NF-kappaB activation through CYLD repression in T cell leukemia. Cancer cell 18: 268-281.
52. Komatsu, K., J. Y. Lee, M. Miyata, J. Hyang Lim, H. Jono, T. Koga, H. Xu, C. Yan, H. Kai, and J. D. Li. 2013 Inhibition of PDE4B suppresses inflammation by increasing expression of the deubiquitinase CYLD. Nature communications 4: 1684.

TABLE 4
Summary of SEQ ID NOS

| SEQ ID NOS | Description |
|---|---|
| 1 | Mouse Perforin-2 cytoplasmic domain |
| 2 | Dog Perforin-2 cytoplasmic domain |
| 3 | Horse Perforin-2 cytoplasmic domain |
| 4 | Human Perforin-2 cytoplasmic domain |

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference.

Although the foregoing invention has been described in some detail by way of illustration and example for purposes of clarity of understanding, it will be obvious that certain changes and modifications may be practiced within the scope of the appended claims.

That which is claimed:

1. A method of treating a subject having inflammation of the gut comprising administering to said subject in need thereof a therapeutically effective amount of a compound that inhibits Perforin-2 activity.

2. The method of claim 1, wherein the subject has colitis.

3. The method of claim 1, wherein the subject has Crohn's disease.

4. The method of claim 1, wherein the subject has inflammatory bowel disease.

5. The method of any one of claims 1-4, wherein the compound comprises: a small molecule, a polypeptide, an oligonucleotide, a polynucleotide or combinations thereof.

6. The method of any one of claims 1-5, wherein the compound that inhibits Perforin-2 activity comprises an inhibitor of at least one component of the ubiquitination pathway.

7. The method of claim 6, wherein the compound that inhibits Perforin-2 activity comprises an E1 ubiquitin-activating enzyme inhibitor, an E2 ubiquitin-conjugating enzyme inhibitor, or an E3 ubiquitin ligase inhibitor.

8. The method of claim 7, wherein the compound that inhibits Perforin-2 activity comprises PYR-41, BAY 11-7082, Nutlin-3, JNJ 26854165, Thalidomide, TAME, NSC-207895, or an active derivative thereof.

9. The method of claim 6, wherein the compound that inhibits Perforin-2 activity comprises a Cullin Ring Ubiquitin Ligase (CRL) inhibitor.

10. The method of claim 5, wherein the compound that inhibits Perforin-2 activity comprises an inhibitor of the neddylation pathway.

11. The method of claim 10, wherein the compound that inhibits Perforin-2 activity comprises a NEDD8-activating enzyme (NAE) inhibitor.

12. The method of claim 11, wherein the NAE inhibitor comprises MLN-4924 or an active derivative thereof.

13. The method of any one of claims 1-5, wherein the compound that inhibits Perforin-2 activity comprises a deamidase.

14. The method of claim 13, wherein the deamidase comprises Cif.

15. The method of any one of claims 1-4, wherein the compound that inhibits Perforin-2 activity comprises a proteasome inhibitor.

16. The method of claim 15, wherein the proteasome inhibitor comprises Bortezomib, Salinosporamide A, Carfilzomib, MLN9708, Delanzomib, or an active derivative thereof.

17. A method of increasing Perforin-2 activity comprising: administering to a subject in need thereof, a therapeutically effective amount of at least one compound which increases the ubiquitination of Perforin-2; and, thereby increasing the activity of Perforin-2.

18. The method of claim 17, wherein the at least one compound increases the activity and/or expression of at least one component of the ubiquitination pathway.

19. The method of claim 18, wherein the at least one component of the ubiquitination pathway comprises an E1 ubiquitin-activating enzyme, an E2 ubiquitin-conjugating enzyme or an E3 ubiquitin ligase.

20. The method of claim 17, wherein the at least one compound comprises an isopeptidase inhibitor.

21. The method of claim 20, wherein said isopeptidase inhibitor comprises Ubiquitin Isopeptidase Inhibitor II (F6) (3,5-bis((4-Methylphenyl)methylene)-1,1-dioxide, piperidin-4-one), Ubiquitin Isopeptidase Inhibitor I (G5) (3,5-bis((4-Nitrophenyl)methylene)-1,1-dioxide, tetrahydro-4H-thiopyran-4-one) or an active derivative thereof.

22. The method of claim 17, wherein the at least one compound comprises a deubiquitinase inhibitor.

23. The method of claim 22, wherein the deubiquitinase inhibitor comprises PR-619, IU1, NSC 632839, P5091, p22077, WP1130, LDN-57444, TCID, b-AP15 or an active derivative thereof.

24. The method of claim 17, wherein the at least one compound comprises a deneddylation inhibitor.

25. The method of claim 24, wherein the deneddylation inhibitor comprises PR-619, Ubiquitin Isopeptidase Inhibitor II (F6) (3,5-bis((4-Methylphenyl)methylene)-1,1-dioxide, piperidin-4-one), Ubiquitin Isopeptidase Inhibitor I (G5) (3,5-bis((4-Nitrophenyl)methylene)-1,1-dioxide, tetrahydro-4H-thiopyran-4-one) or an active derivative thereof.

26. The method of any one of claims 17-25, wherein the at least one compound inhibits replication, inhibits growth, or induces death of an infectious disease organism.

27. The method of claim 26, wherein the infectious disease organism is an intracellular bacterium.

28. A method of treating a subject suffering from an infectious disease organism comprising, administering to the subject a therapeutically effective amount of at least one compound that increases the activity of Perforin-2, wherein said compound increases the ubiquitination of Perforin-2.

29. The method of claim 28, wherein the at least one compound increases the activity or expression of at least one component of the ubiquitination pathway.
